(19) United States
(12) Patent Application Publication
(10) Pub. No.: US 2024/0374879 A1
(43) Pub. Date: Nov. 14, 2024

(54) METHODS AND COMPOSITIONS TO REDUCE CELLULAR DEPOSITION, AND HYDROCEPHALUS SHUNT FAILURE

(71) Applicant: Wayne State University, Detroit, MI (US)

(72) Inventors: Carolyn Harris, Detroit, MI (US); Mira Zaranek, Detroit, MI (US); Fatemeh Khodadadei, Detroit, MI (US)

(73) Assignee: Wayne State University, Detroit, MI (US)

(21) Appl. No.: 18/690,241

(22) PCT Filed: Sep. 9, 2022
(86) PCT No.: PCT/US2022/076241

§ 371 Date: Mar. 7, 2024

Related U.S. Application Data
(63) Continuation-in-part of application No. PCT/US2022/076236, filed on Sep. 9, 2022.
Application No. PCT/US2022/076236 is a continuation-in-part of application No. PCT/US2022/076230, filed on Sep. 9, 2022.
(60) Provisional application No. 63/242,468, filed on Sep. 9, 2021.

(51) Int. Cl. A61M 27/00; A61K 31/42

(52) U.S. Cl. CPC A61M 27/006; A61K 31/42; A61M 2205/0238; A61M 2205/04; A61M 2210/0693

(57) ABSTRACT

This disclosure provides compositions, devices, systems, and methods that reduce the likelihood, amount, or level of cellular deposition (and associated blockage and/or failure) of implantable medical devices, such as central nervous system implants. Embodiments involve use of inhibitor(s) of one or more of TLR-4, TNF-α, IL-1β, or IL-6, for instance, to prevent, reduce, or reverse activation of astrocyte and/or glia cells, and/or to prevent, reduce, or reverse attachment of such cells to the surface of a medical device in contact with a biological fluid, such as cerebrospinal fluid.

### CROSS-REFERENCE TO RELATED APPLICATIONS

This application is the 371 National Phase of International Application No. PCT/US2022/076241, filed on Sep. 9, 2022, which claims priority to PCT Application No. PCT/US2022/076230 and PCT Application No. PCT/US2022/076236, both of which were filed Sep. 9, 2022; and priority to and the benefit of the earlier filing of U.S. Provisional Application No. 63/242,468, filed on Sep. 9, 2021. Each of these applications is incorporated by reference herein in its entirety (including Exhibits C-E of U.S. Provisional Application No. 63/242,468).

### STATEMENT REGARDING FEDERALLY SPONSORED RESEARCH OR DEVELOPMENT

This invention was made with government support under Grant No. 5R01 NS094570-05 awarded by the National Institutes of Health (National Institute of Neurological Disorders and Stroke; NINDS). The government has certain rights in the invention.

### FIELD OF THE DISCLOSURE

The present disclosure relates generally to compositions, methods, and devices, and more specifically, to compositions and methods that reduce the likelihood or severity of cellular deposition and/or blockage associated with a medical implant. This disclosure further relates to improvements in systems, system elements, and methods related to fluid flow and circulation of fluids, including flow of biological fluids in organs or tissues, such as cerebrospinal fluid in brain tissue.

### BACKGROUND OF THE DISCLOSURE

Hydrocephalus, an imbalance between cerebrospinal fluid (CSF) production and absorption, is diagnosed in more than 1 in 500 people in the United States. Approximately 80% of these patients will suffer long-term neurological deficits. Genetic diseases, meningitis, subarachnoid hemorrhage, stroke, traumatic brain injury, or tumors, cause hydrocephalus.

The common treatment for all hydrocephalus patients is CSF drainage by shunting. Despite all efforts to date, shunts still have the highest failure rate of any neurological device. A shocking 98% of shunts fail after just ten years, a rate bumped up by the 80% of patients who suffer from tens if not hundreds of repetitive shunt failures. Shunts fail after becoming obstructed with attaching glia, creating a substrate for more glia or other cells and tissues (e.g. choroid plexus) to secondarily bind and block the flow of CSF through the shunt. For additional discussion, see US Patent Publication No. 2012/0060622 (Harris et al.).

Hydrocephalus patients can have a diminished quality of life and suffer from long-term neurologic deficits because of the failure of current treatments in the field, most of which involve diversion of cerebrospinal fluid (CSF) with shunts. Despite our efforts for nearly seven decades, shunts still have the highest failure rate of any neurological device: 98% of all shunts fail after ten years. This failure rate is the dominant contributor to the $2 billion-per-year cost that hydrocephalus incurs on our health care system.

While many factors such infection and disconnection could lead shunt obstruction and eventual failure, the statistics tell us that most shunts fail by becoming blocked with cells and tissues. But how does this happen? The literature predicts that there are four mechanisms: (1) cells coming from the brain, attaching and blocking the ventricular catheter; (2) cells, protein and debris from the CSF attaching and blocking the ventricular catheter; (3) blockage by the ventricular catheter laying on the ventricular wall's epithelial cells; (4) blockage by the choroid plexus (lined with epithelial cells).

Shunts are far from ideal even when they are not occluded, and patients often experience discomfort such as headaches and pain on a regular basis which directly impact their quality of life. While valves are necessary components of a shunt system, their outdated design results in sudden pressure changes that have been shown to significantly increase the rate of shunt failure through at least three of the mechanisms mentioned earlier. In addition, regular valves offer no protection against over-drainage and under-drainage; two of the most common causes of serious conditions including but not limited to chronic headache hemorrhage

There is an ongoing urgent need to improve hydrocephalus treatment.

### SUMMARY OF THE DISCLOSURE

Provided herein in one embodiment is an implantable medical device including: a coating including a biologically active agent over at least a portion of the surface of the device that is exposed to a biological fluid when the device is implanted, wherein the biologically active agent includes: a neutralizing antibody specific for TLR-4, IL-1β, IL-6, TNF-α, IL-1α, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Provided herein in another embodiment is a medical implant that releases a biologically active agent in an amount effective to reduce or inhibit astrocyte and/or glia cell deposition associated with the medical implant, wherein the biologically active agent is selected from the group consisting of: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ, and wherein the medical implant includes 0.1 μg to 1 mg of biologically active agent per mm² of surface area of the portion of the medical implant to which the biologically active agent is applied or incorporated. In examples of this embodiment, the medical implant includes one or more of a tube, a chronic indwelling central nervous system (CNS) catheter, a neurological or neurosurgical device, a central nervous shunt, a pump, or a catheter.

Also provided is a ventricular shunt coated with GIT 27 or another TLR-4 inhibitor.

Provided herein in another embodiment is an improved shunt catheter system, the improvement including in the system at least one biologically active agent selected from the group consisting of: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

In examples of the improved shunt catheter system, the inhibitor of production or activity of TNF-α includes one or more of: a neutralizing antibody specific for TNF-α, etanercept, infliximab, adalimumab, certolizumab pegol, or golimumab. In other embodiments of the shunt catheter system, the inhibitor of production or activity of IL-1α or IL-1β includes one or more of: a neutralizing antibody for IL-1, a neutralizing antibody specific for IL-1α, a neutralizing antibody specific for IL-1β, anakinra, Rilonacept, or canakinumab. In yet additional embodiments of the improved shunt catheter system, the inhibitor of production or activity of TLR-4 includes one or more of: a neutralizing antibody of TLR-4, GIT 27, Eritoran, ibudilast, NI-0101, 1A6, or 15C1. And yet more embodiments of the improved shunt catheter system, the inhibitor of production of IL-6 includes one or more of Tocilizumab, Sarilumab, Clazakizumab, Olokizumab, ALX-006,

Provided herein in another embodiment is a method of reducing cellular deposition and/or shunt device blockage in a fluid moving system, including introducing into the system: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Also provided is a method of reducing shunt device failure, including: inhibiting astrocyte and/or glia cell activation by contacting the shunt and/or a fluid passing over/through the shunt with: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Yet another embodiment is a method of mitigating astrocyte immune response to a chronically indwelling neuroprosthetic device, including: inhibiting secretion and/or activity of at least one of TNF-α, IL-1β, or IL-6; or inhibiting secretion and/or activity of TLR-4, TLR2/6, or IFN-γ.

In examples of any of the provided method embodiments, the method inhibits formation or reverses formation of glial scar.

In examples of any of the provided method embodiments, the method inhibits obstruction of one or more openings in the device.

Also provided is a method of reducing astrocyte activation and attachment on a surface of a shunt, including contacting the shunt surface with a composition including: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Yet another embodiment is a method of reducing obstruction/blockage and failure of a ventricular shunt in a hydrocephalus patient, including contacting the shunt, before or after installation of the shunt in the patient, with a composition including: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Another provided embodiment is a method to decrease cellular attachment to a catheter surface, including contacting a surface of the catheter with a composition including: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Also described are methods of decreasing astrocyte cell attachment on a medical implant device, including contacting the device with GIT 27 or another TLR-4 inhibitor before installation of the medical implant device.

Yet another embodiment is a method of decreasing secretion of cytokines from astrocyte cells in a neurological implant system, including contact the astrocyte cells with GIT 27 or another TLR-4 inhibitor during or after installation of a neurological implant device.

Also provided herein are devices, methods, systems, and compositions for inhibiting or reducing cellular deposition on a medical implant, essentially as described herein.

### BRIEF DESCRIPTION OF DRAWINGS

FIG. 1 is a schematic representation of microglia/macrophage and astrocyte reactions following neuroprosthetic device implantation. TNF-α=solid triangle; IL-1α

FIGS. 2A-2B. Expression of C3, EMP1 astrocyte activation genes assessed by qPCR on non-obstructed and obstructed shunts. FIG. 2A shows heatmaps comparing the expression of A1-specific reactive gene C3 and the A2-specific reactive gene EMP1 for non-obstructed (top) and obstructed (bottom) shunts collected from patients (two-way ANOVA test). FIG. 2B shows representative images for non-obstructed (top) and obstructed (bottom) shunts.

FIGS. 3A-3B. Comparison of astrocyte response by RNAscope fluorescent in situ hybridization on obstructed and non-obstructed shunts. FIG. 3A shows the astrocyte phenotype specificity of C3 and EMP1 RNAscope fluorescent in situ hybridization signal was assessed by probing for SLC1A3+ astrocytes on both obstructed and non-obstructed shunts collected from patients. For normalization, the C3 and EMP1 signals were dividing by SLC1A3 signals (*p<0.05; two-way ANOVA test). FIG. 3B shows representative RNAscope fluorescent in situ hybridization images for obstructed and non-obstructed astrocyte gene C3 and EMP1 showing colocalization with the astrocyte marker SLC1A3 (scale bar=500 μm).

FIGS. 4A-4H. Cerebrospinal fluid cytokine concentrations between obstructed and non-obstructed shunts. Analytes include C3 (FIG. 4A), C1q (FIG. 4B), and IL-1a (FIG. 4C) (A1 astrocyte markers), IL-1p (FIG. 4D) and IL-6 (FIG. 4F) (A2 astrocyte markers), TNF-α (FIG. 4E), IL-8 (FIG. 4G) and IL-10 (FIG. 4H). For normalization, the concentration of each cytokine is divided by the total protein concentration for each group (two-tailed unpaired Student's t-test, n=10 per group, mean±SEM).

FIGS. 5A-5B Antibody therapies that will inhibit the cell activation state to reduce attachment on the shunt surface. FIG. 5A shows results from A1 reactive astrocytes treated with neutralizing antibodies to TNF-α, IL-1α (left graph) and anti-inflammatory cytokine TGF-β (right graph). FIG. 5B shows results from A2 reactive astrocytes treated with neutralizing antibodies to TNF-α, IL-1β (left graph), and IL-6 (right graph) (*p<0.05; two-tailed unpaired Student's t-test,n=3 per group, mean±SD).

FIGS. 6A-6F Visual representation of the treatment timelines and composition of media solution for the indicated experiments: Pre-IL1B (FIG. 6A), Sim-IL1B (FIG. 6B), Post-IL1B (FIG. 6C), Pre-IL10 (FIG. 6D), Sim-IL10 (FIG. 6E), Post-IL10 (FIG. 6F). See Example 2 for additional details.

FIGS. 7A-7B Pre-Treatment Data Comparisons by Group. Comparisons of samples from the three treatment groups when under pre-treatment conditions. FIG. 7A shows Cell Counts of Samples Exposed to a Pre-Treatment of GIT 27; FIG. 7B shows Cytokine Concentration of Samples Exposed to a Pre-Treatment of GIT 27.

FIGS. 8A-8B Simultaneous Treatment Data Comparisons by Group. Comparisons of samples from the three treatment groups when under simultaneous treatment conditions. FIG. 8A Cell Counts of Samples Exposed to a Simultaneous Treatment of GIT 27. FIG. 8B Cytokine Concentration of Samples Exposed to a Simultaneous Treatment of GIT 27.

FIGS. 9A-9B Post-Treatment Data Comparisons by Group. Comparisons of samples from the three treatment groups when under post-treatment conditions. FIG. 9A Cell Counts of Samples Exposed to a Post-Treatment of GIT 27. FIG. 9B Cytokine Concentration of Samples Exposed to a Post-Treatment of GIT 27.

FIGS. 10A-10B Treatment Time Point Comparisons of Vehicle Group. Comparisons of samples from the three treatment times when treated with the vehicle control DMSO. FIG. 10A Cell Counts of Samples Exposed to DMSO vehicle at the Three Treatment Times. FIG. 10B Cytokine Concentration of Samples Exposed to DMSO vehicle at the Three Treatment Times.

FIGS. 11A-11B Treatment Time Point Comparisons of Vehicle Group. Comparisons of samples from the three treatment times when treated with GIT 27. FIG. 11A Cell Counts of Samples Exposed to GIT 27 at the Three Treatment Times. FIG. 11B Cytokine Concentration of Samples Exposed to GIT 27 at the Three Treatment Times.

FIGS. 12A-12D Peeling vs Sluffing. Visual example of samples that are confluent (FIG. 12A), bare (FIG. 12B), sluffing off (FIG. 12C), or peeling off (FIG. 12D).

FIG. 13 illustrates a horizontal head computed tomography (CT) image showing a catheter tip implanted in a patient's brain.

FIG. 14 illustrates an example shunt system.

### DETAILED DESCRIPTION

The common treatment for all hydrocephalus patients is CSF drainage by shunting. Despite all efforts to date, shunts still have the highest failure rate of any neurological device. A shocking 98% of shunts fail after just ten years, a rate bumped up by the 80% of patients who suffer from tens if not hundreds of repetitive shunt failures. Shunts fail after becoming obstructed with attaching glia, creating a substrate for more glia or other cells and tissues (e.g. choroid plexus) to secondarily bind and block the flow of CSF through the shunt. For additional discussion, see US Patent Publication No. 2012/0060622 (Harris et al.), and WO 2022/016072 (Horbatiuk et al.).

The present disclosure provides compositions and methods for preventing, reducing or inhibiting the likelihood of cellular deposition associated with medical implants.

This disclosure provides compositions, devices, systems, and methods that reduce the likelihood, amount, or level of cellular deposition (and associated blockage and/or failure) of implantable medical devices, such as central nervous system implants. Embodiments involve use of inhibitor(s) of one or more of TLR-4, TNF-α, IL-1α, or IL-1p to prevent, reduce, or reverse activation of astrocyte and/or glia cells, and/or to prevent, reduce, or reverse attachment of such cells to the surface of a medical device in contact with a biological fluid, such as cerebrospinal fluid.

More specifically, in one aspect medical implants or devices are provided which release a therapeutic agent, wherein the therapeutic agent reduces, inhibits, or prevents attachment of astrocytes or glia cells in contact with or are associated with the medical device or implant. For example, within one aspect of the disclosure, medical implant or devices are provided which release a biologically active agent, such as for instance neutralizing antibody specific for TLR-4, TNF-α, IL-1α, or IL-1β, or an inhibitor of the product or an activity of TLR-4, TNF-α, IL-1α, or IL-1β. Within various embodiments, the implant is coated in whole or in part with a composition comprising a neutralizing antibody specific for or an inhibitor of the production or activity of one of TLR-4, TNF-α, IL-1α, or IL-1β.

Aspects of the present disclosure provide methods for making medical implants, comprising adapting a medical implant (e.g., coating the implant) with a composition comprising one or more of a neutralizing antibody specific for TLR-4, TNF-α, IL-1α, or IL-1β, or an inhibitor of the product or an activity of TLR-4, TNF-α, IL-1α, or IL-1β.

A wide variety of medical implants can be generated using the methods provided herein, including for example, catheters, shunts, tubes, valves, and so forth. Central nervous system (CNS) shunts and catheters are particularly contemplated, including indwelling devices such as medical devices used to tread or moderate hydrocephalus. Specifically contemplated are shunt catheters.

Within further aspects of the disclosure, there is provided a shunt which releases a biologically active agent selected from the group consisting of a neutralizing antibody specific for TLR-4, TNF-α, IL-1α, or IL-1β, or another inhibitor of the production or an activity of TLR-4, TNF-α, IL-1α, or IL-1β. In one embodiment, the inhibitor of production or activity of TNF-α comprises one or more of: etanercept, infliximab, adalimumab, certolizumab pegol, or golimumab. In another embodiment, the inhibitor of production or activity of IL-1α or IL-1p comprises one or more of: anakinra, Rilonacept, or canakinumab. In yet another embodiment, the inhibitor of production or activity of TLR-4 comprises one or more of: GIT 27, Eritoran, ibudilast, NI-0101, 1A6, or 15C1. In other embodiments, the shunt further comprises a polymer wherein the biologically active agent is released from a polymer on the shunt.

Also provided are methods for reducing or inhibiting cellular deposition (e.g., deposition of astrocytes and/or glial cells) associated with a medical implant, comprising the step of introducing into a patient a medical implant which has been contacted or coated with a neutralizing antibody specific for TLR-4, TNF-α, IL-1α, or IL-1β, or another inhibitor of production or an activity of TLR-4, TNF-α, IL-1α, or IL-1β.

Also provided are methods to decrease cellular attachment to a catheter surface, such as a surface of a catheter implanted in the CNS of a subject. By way of example, embodiments of this method provide a method to decrease cellular attachment to a catheter surface, which method includes contacting the surface (or a fluid that is passed over or by the surface) with GIT 27 or another compound that reduces activity or production of one or more pro-inflammatory cytokines (e.g., TLR4, TLR2/6, IL-1β, IL-10, and/or IFN-γ). In some instances, the fluid comprises cerebrospinal fluid (CSF). In some instances, the fluid contains at least one neutralizing antibody(s) against at least one cytokine(s) (e.g., TLR-4, TNF-α, IL-1α, or IL-1β). In some instances, the fluid comprises a chemical inhibitor; by way of example, the chemical inhibitor may comprise an inhibitor of TLR-4, such as GIT 27.

Provided herein is an implantable medical device including: a coating including a biologically active agent over at least a portion of the surface of the device that is exposed to a biological fluid when the device is implanted, wherein the biologically active agent includes: a neutralizing antibody specific for TLR-4, IL-1β, IL-6, TNF-α, IL-1α, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Also provided herein is a medical implant that releases a biologically active agent in an amount effective to reduce or inhibit astrocyte and/or glia cell deposition associated with the medical implant, wherein the biologically active agent is selected from the group consisting of: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ, and wherein the medical implant includes 0.1 μg to 1 mg of biologically active agent per mm² of surface area of the portion of the medical implant to which the biologically active agent is applied or incorporated.

Another embodiment is a ventricular shunt coated with GIT 27 or another TLR-4 inhibitor.

Provided herein in another embodiment is an improved shunt catheter system, the improvement including in the system at least one biologically active agent selected from the group consisting of: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Another provided embodiment is a method of reducing cellular deposition and/or shunt device blockage in a fluid moving system, including introducing into the system: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Also provided is a method of reducing shunt device failure, including: inhibiting astrocyte and/or glia cell activation by contacting the shunt and/or a fluid passing over/through the shunt with: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Yet another embodiment is a method of mitigating astrocyte immune response to a chronically indwelling neuroprosthetic device, including: inhibiting secretion and/or activity of at least one of TNF-α, IL-1β, or IL-6; or inhibiting secretion and/or activity of TLR-4, TLR2/6, or IFN-γ.

Also provided is a method of reducing astrocyte activation and attachment on a surface of a shunt, including contacting the shunt surface with a composition including: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Yet another embodiment is a method of reducing obstruction/blockage and failure of a ventricular shunt in a hydrocephalus patient, including contacting the shunt, before or after installation of the shunt in the patient, with a composition including: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Another provided embodiment is a method to decrease cellular attachment to a catheter surface, including contacting a surface of the catheter with a composition including: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

Also described are methods of decreasing astrocyte cell attachment on a medical implant device, including contacting the device with GIT 27 or another TLR-4 inhibitor before installation of the medical implant device.

Yet another embodiment is a method of decreasing secretion of cytokines from astrocyte cells in a neurological implant system, including contact the astrocyte cells with GIT 27 or another TLR-4 inhibitor during or after installation of a neurological implant device.

In examples of any of the embodiments provided herein, the inhibitor of production or activity of TNF-α may comprise one or more of: a neutralizing antibody specific for TNF-α, etanercept, infliximab, adalimumab, certolizumab pegol, or golimumab; the inhibitor of production or activity of IL-1α or IL-1p may comprise one or more of: a neutralizing antibody for IL-1, a neutralizing antibody specific for IL-1α, a neutralizing antibody specific for IL-1β, anakinra, Rilonacept, or canakinumab; the inhibitor of production or activity of TLR-4 may comprise one or more of: a neutralizing antibody of TLR-4, GIT 27, Eritoran, ibudilast, NI-0101, 1A6, or 15C1; and the inhibitor of production of IL-6 may comprise one or more of Tocilizumab, Sarilumab, Clazakizumab, Olokizumab, ALX-006, or 116-25.

Also provided herein are devices, methods, systems, and compositions for inhibiting or reducing cellular deposition on a medical implant, essentially as described herein.

Aspects of the current disclosure are now described with additional details and options as follows: (I) Hydrocephalus Crisis in Care; (II) Reducing Cell Deposition on Implants; (III) Composition & Formulations; (IV) Medical Implants; (V) Clinical Applications; (VI) Shunt Systems Comprising a Catheter; (VII) Kits; (VIII) Exemplary Embodiments; (IX) Experimental Examples; and (X) Closing Paragraphs. These headings do not limit the interpretation of the disclosure and are provided for organizational purposes only.

### I. Hydrocephalus Crisis in Care

Over the past decade, there has been an increase in the diagnosis of hydrocephalus and an increase in hydrocephalus prevalence in children and adults, but there have been few attempts to improve treatment, and no improvements in shunt design that have improved patient care. Hydrocephalus, an accumulation of cerebrospinal fluid in the ventricles of the brain, is: (1) as common as Down's syndrome; (2) is caused by many pathological states like premature birth, traumatic brain injury, stroke, meningitis, and age; and (3) leads to long-term neurological deficits in 80% of patients. The overwhelming majority of patients are treated using a shunt to drain excess cerebrospinal fluid (CSF) from the cerebral ventricles, but a shocking 85% of these shunts fail, leading to a necessity for shunt revision. Shunts are composed of two polydimethylsiloxane (PDMS, silicone) shunt catheters connected by a pressure valve. One catheter remains in the ventricles, while the other is tunneled subcutaneously into the peritoneum or atrium.

Why Hydrocephalus Treatment Fails: Contact and Growth Hydrocephalus treatment fails most often because the outflow pathway created by the holes in the shunt's ventricular catheter become obstructed with tissue. Up until a few years ago, the most significant studies on shunt failure revealed that shunts most commonly harbor inflammatory glia, lymphocytic inflammation, and foreign body giant cells. Our work shows that the tissue occluding shunts is predominately composed of astrocytes and macrophages, has only sparse microglia, has more activated cells on obstructed shunts than unobstructed, stain positive for proliferative markers, has reactivity that follows flow, and predominately obstructs shunts as large tissue masses. We have also seen that this cellular response occurs proportionally to length of time implanted and non-uniformly related to the number of shunt revisions a patient has undergone. This is suggestive of a dynamic, evolving, cellular active ventricular environment.

Contact Identifying the source of these cells bound to the shunt catheter may seem intuitive: cells are likely born from the tissues that surround the shunt catheter. Existing neurosurgical lore would suggest this intuition to be true: it is implied that these cells come from tissue contact (i.e. physical contact with the ventricular wall, parenchyma, or choroid plexus), tissue contact plus an active cell growth response (i.e. ventricular wall contact and ingrowth), or an active growth without any tissue contact (i.e. cells in CSF binding and proliferating). One of the most groundbreaking studies has shown that tissue contact is correlated to dynamic ventricular size change from over-drainage. Knowing that the dynamic environment of the hydrocephalic ventricle may play a role in obstruction, the next logical step is to clearly define causation between tissue contact and the single or repetitive events that may contribute to dynamic ventricular size change.

Key relationships between clinical factors of hydrocephalus, cell activation, cell proliferation, and cell growth onto the shunt are not well understood. For instance, ventriculomegaly is known to increase cell shedding into the CSF, but once those cells contact the shunt catheter, what in the CSF triggers them to actively bind and proliferate? How does that active binding and proliferation change with dynamic changes in the ventricular environment? It is known that cells on obstructed shunts are classically (vs. alternatively) activated, but what conditions of single or repetitive tissue contact preceded that activation, and to what degree does that play a role in causing the obstruction?

### II. Reducing Cell Deposition on Implants

Provided herein are advances that enable clinically-relevant inhibition of cellular deposition (such as deposition of astrocytes and/or glia cells) on medical devices and implants. This disclosure provides medical implants (as well as compositions and methods for making and using medical implants), such as central nervous system implants, with reduced likelihood of or amount/level of cellular deposition (and associated blockage and/or failure). Cellular deposition is a common complication of the implantation of foreign bodies such as medical devices.

The phrase “medical implant” refers to devices or objects that are implanted or inserted into a body. Representative examples include catheters, tubes, shunts, and so forth. Particularly contemplated are neural implants, such as chronically indwelling implants and prosthetics, including central nervous system medical devices and implants. Specific examples include cerebral shunts and catheters, such as ventricular catheters and other components of systems used to manage or treat hydrocephalus.

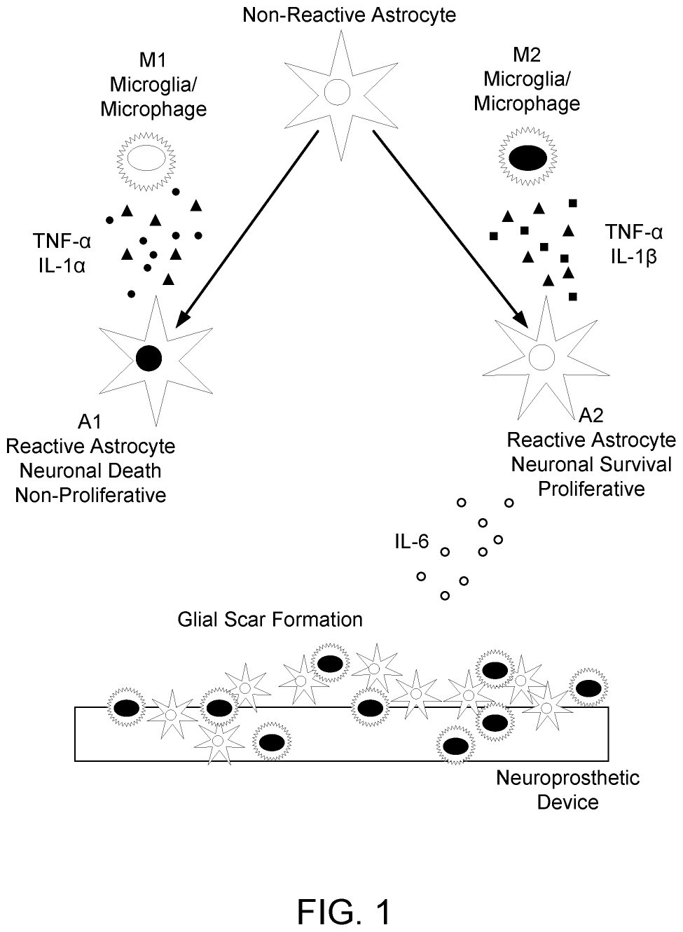

Described herein is a precise understanding of cellular response mechanism to device implantation and a precise interpretation of failure in chronically indwelling neuroprosthetic implants, which enables targeted therapies to inhibit astrocyte activation and attachment (more generally, cellular deposition) on the implant. Injury such as incurs upon insertion of an implant transforms microglia into an M1- and M2-like phenotype and astrocytes into an A1- and A2-type, correspondingly (see FIG. 1). Astrocytes and microglia work together to initiate either a neuroinflammatory or neuroprotective response after injury, through the release of cytokines or neurotrophic factors that can lead to neuronal death or survival. The cytokine pathway is the best and most important measurable outcome for inflammatory cascades. Inflammatory cells at the brain-device interface communicate via cytokines to activate and recruit other inflammatory cells to the interface. Cytokine stimulation is a gateway for other gene products to be over- or under-expressed in the cascade, resulting in implant device failure.

As detailed in Example 1, pharmacological agents that inhibit cell activation (e.g., astrocyte and/or microglia cells) can reduce the presence of astrocytes on CNS shunts. This can keep any attaching astrocytes in a resting state, reducing proliferation, inhibiting downstream proliferation, and ultimately deterring shunt obstruction and failure. Therefore, for significant reduction in device failure, the herein provide drug therapies can be used for inhibition of cytokines and therefore inhibition of cell aggregation for achieving stable and long-term functional outcomes.

The master cytokine IL-1 (both α and β) is the initial molecular mediator that triggers glial scar formation around implanted devices in the brain. As described herein, astrocyte obstruction of shunts could be prevented by blocking secretion or action of these cytokines—thereby keeping astrocytes out of the A1 or A2 reactive state. Specifically contemplated herein is use of biologically active agents that target any of TNF-α, IL-1α, IL-1β, and/or IL-6 to inhibit astrocyte and/or glia cell depositions on CNS implants. In embodiments, it is particularly desired to inhibit or minimize activity of those compounds involved in the A2 cascade (see FIG. 1)—that is, TNF-α, IL-1β, and/or IL-6.

For instance, neutralizing antibodies to one of these cytokines can be used to reduce or inhibit their activity—and thereby reduce cellular deposition and glial scar formation. As demonstrated in Example 1, neutralizing antibodies to TNF-α, IL-1α, IL-13 and IL-6 can be used to reduce astrocyte activity. In embodiments, it is particularly desired to use neutralizing antibodies specific for compounds involved in the A2 cascade (see FIG. 1)—that is, TNF-α, IL-1β, and/or IL-6.

Additional representative agents that block secretion and/or action of TNF-α are believed to be useful in methods and devices described herein, including for instance Tumor Necrosis Factor (TNF)-α inhibitors, such as etanercept (Moreland et al., Ann Intern Med. 130(6):478-86, 1999), infliximab (Cheifetz et al., Am J Gastroenterol. 98(6):1315-24, 2003; Chung et al., Circulation. 107(25):3133-40, 2003), adalimumab (Oberoi et al., PLoS One. 11(7):e0160145, 2016. doi: 10.1371/journal.pone.0160145), certolizumab pegol (Nesbitt et al., Inflamm BowelDis. 13(11):1323-1332, 2007. doi:10.1002/ibd.20225), and golimumab (Ono et al., Protein Sci. 27(6):1038-1046, 2018. doi: 10.1002/pro.3407).

Additional representative agents that block secretion and/or action of IL-1 and IL-1α are believed to be useful in methods and devices described herein, including for instance canakinumab (human monoclonal anti-IL-1p antibody; see Brucato et al., JAMA. 316(18):1906-1912, 2016. doi:10.1001/jama.2016.15826), anakinra (Kineret®; recombinant human IL-1Ra; inhibits activity of IL-1α and IL-1β by inhibiting IL-1 binding to IL-1 type I receptor; Cvetkovic & Keating, BioDrugs 16(4):301-311, 2002), and rilonacept (a soluble decoy receptor ‘trap’, binding both IL-1α and IL-1β; Klein et al., Am Heart J228:81-90, 2020).

Additional representative agents that block secretion and/or action of IL-6 are believed to be useful in methods and devices described herein, including for instance IL-6 inhibitors, such as Tocilizumab (Mihara et al., Open Access Rheumatol. 3:19-29, 2011. doi: 10.2147/OARRR.S17118), Sarilumab (Rafique et al., Ann Rheum Dis. Annals of the Rheumatic Diseases. 72:A797, 2013), Clazakizumab (Zhao et al., Arthritis Rheum. 65 Suppl:S1020, 2013; and Jordan et al., Kidney Int Rep. 7(4):720-731, 2022. doi:10.1016/j.ekir.2022.01.1074), Olokizumab (Nasonov et al., Ann Rheum Dis. 2022; 81(4):469-479. doi:10.1136/annrheumdis-2021-219876), ALX-0061 (Van Roy et al., Arthritis Res Ther. 17(1): 135, 2015. doi:10.1186/s13075-015-0651-0), and Sirukumab (Smolen et al. Ann Rheum Dis. 73(9):1616-25, 2014. doi: 10.1136/annrheumdis-2013-205137).

Also described herein is the discovery that GIT 27, an inhibitor of TLR-4, is effective to reduce cellular attachment such as that which occurs in cellular deposition on CNS implants, such as shunts and catheters. Thus, there are enabled herein methods of using GIT 27, or another immunomodulator that reduces production of pro-inflammatory cytokines, including particularly compounds that inhibit TNF-α, and/or interferes with TLR4 and/or TLR2/6, and/or that reduces secretion of one or more of IL-1β, IL-10 and IFN-γ, to reduce cellular deposition on indwelling medical devices such as CNS implants.

GIT 27 (4,5-Dihydro-3-phenyl-5-isoxazoleacetic acid) is an orally active immunomodulatory agent that primarily targets macrophages; it inhibits TNF-α secretion via interference of macrophage toll-like receptor (TLR) 4 and TLR 2/6 signaling pathway and reduces the secretion of pro-inflammatory cytokines IL-1β, IL-10 and IFN-γ. GIT 27 (4,5-Dihydro-3-phenyl-5-isoxazoleacetic acid, a.k.a. 2-(3-phenyl-4,5-dihydro-1,2-oxazol-5-yl)acetic acid; PubChem CID 10798271) (available for Tocris Bioscience) is an orally active immunomodulatory agent that primarily targets macrophages. It inhibits TNF-α secretion via interference of macrophage toll-like receptor (TLR) 4 and TLR 2/6 signaling pathway. It also reduces secretion of pro-inflammatory cytokines IL1-β, IL-10 and IFN-γ. See Saurus et al. (Cell Death Dis 6(5):e1752, 2015 doi:10.1083/cdds.2015.125); Stosic-Grujicic et al. (J. Pharmacol. Exp. Ther. 320 1038, 2007); Stojanovic et al. (Clin. Immunol. 123 311, 2007); Mangano et al. (Eur. J. Pharmacol. 586 313, 2008). The following is the chemical structure of GIT 27:

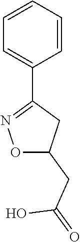 [SMILES: O=C(O)CC1CC(c2ccccc2)=NO1]

GIT 27 has immunomodulatory properties that interfere with the signaling process associated with TLR-4. In the study described in Example 2, it was demonstrated that treatment using GIT 27 is suspension was effective for reducing cell attachment, including in an in vitro system mimicking aspects of the injury associated with shunt insertion into the brain. Moreover, GIT 27 treatment in the described in vitro system causes a majority of the decrease in the overall cytokine response. GIT 27 had the greatest effect on the cell count at the pre-treatment timepoint (analogous to application of the GIT 27 before insertion of the implant into a subject). GIT 27 post-treatment appears to provide the largest effect on cytokine concentration (while at the same timepoint, DMSO has the smallest effect on cytokine concentration).

Additional representative agents that inhibit TLR-4 are known in the art; see, for instance, Hennessy et al. (Nat Rev Drug Discov 9:293-307, 2010. https://doi.org/10.1038/nrd3203). For instance, the following additional TLR4 inhibitors are contemplated: Eritoran (E5564; Mullarkey et al., J. Pharmacol. Exp. Ther. 304:1093-1102, 2003; Savov et al., Am. J. Physiol. Lung Cell. Mol. Physiol. 289, L329-L337, 2005); Ibudilast (Ledeboer et al., Neuron Glia Biol. 2:279-291, 2006; Ledeboer et al., Expert Opin. Investig. Drugs 16, 935-950, 2007); NI-0101 (Monnet et al., Clin Pharmacol Ther. 101(2):200-208, 2017. doi:10.1002/cpt.522); 1A6 (Ungaro et al., Am. J. Physiol. Gastrointest Liver Physiol. 296, G1167-G1179, 2009); and 15C1 (Dunn-Siegrist et al., J. Biol. Chem. 282:34817-34827, 2007).

### III. Composition & Formulations

Therapeutic agents described herein may be formulated in a variety of manners, and may additionally include carrier(s). In this regard, a wide variety of carriers may be selected of either polymeric or non-polymeric form. The polymers and non-polymer-based carriers and formulations that are discussed in more detail below are provided merely by way of example.

A wide variety of polymers can be utilized to contain and/or deliver one or more of the biologically active agents discussed herein, including for example biodegradable and non-biodegradable compositions. Representative examples of biodegradable compositions include albumin, collagen, gelatin, chitosan, hyaluronic acid, starch, cellulose and derivatives thereof (e.g., methylcellulose, hydroxypropylcellulose, hydroxypropylmethylcellulose, carboxymethyl-cellulose, cellulose acetate phthalate, cellulose acetate succinate, hydroxypropylmethylcellulose phthalate), alginates, casein, dextrans, polysaccharides, fibrinogen, poly(L-lactide), poly(D,L lactide), poly(L-lactide-co-glycolide), poly(D,L-lactide-co-glycolide), poly(glycolide), poly(tri-methylene carbonate), poly(hydroxyvalerate), poly(hydroxybutyrate), poly(caprolactone), poly(alkylcarbonate) and poly(orthoesters), polyesters, poly(hydroxyvaleric acid), polydioxanone, poly(malic acid), poly(tartronic acid), polyanhydrides, polyphosphazenes, poly(amino acids), copolymers of such polymers and blends of such polymers (see generally, Ilium, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986).

Representative examples of nondegradable polymers include poly(ethylene-co-vinyl acetate) (“EVA”) copolymers, silicone rubber, acrylic polymers (e.g., polyacrylic acid, polymethylacrylic acid, poly(hydroxyethylmethacrylate), polymethylmethacrylate, polyalkyl-cyanoacrylate), polyethylene, polyproplene, polyamides (e.g., nylon 6,6), polyurethane (e.g., poly(ester urethanes), poly(ether urethanes), poly(ester-urea), poly(carbonate urethanes)), polyethers (e.g., poly(ethylene oxide), poly(propylene oxide), PLURONICS® and poly(tetramethylene glycol)) and vinyl polymers [e.g., polyvinylpyrrolidone, poly(vinyl alcohol), poly(vinyl acetate phthalate)]. Polymers may also be developed which are either anionic (e.g., alginate, carrageenin, carboxymethyl cellulose and poly(acrylic acid), or cationic (e.g., chitosan, poly-L-lysine, polyethylenimine, and poly (allyl amine)) (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int′l J. Pharm. 120:115-118, 1995; Miyazaki et al., Int′l J. Pharm. 118:257-263, 1995). Exemplary polymeric carriers include poly(ethylene-co-vinyl acetate), polyurethane, acid, poly(caprolactone), poly(valerolactone), polyanhydrides, copolymers of poly(caprolactone) or poly(lactic acid) with a polyethylene glycol (e.g., MePEG), and blends thereof.

Other representative polymers include carboxylic polymers, polyacetates, polyacrylamides, polycarbonates, polyethers, polyesters, polyethylenes, polyvinylbutyrals, polysilanes, polyureas, polyurethanes, polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxy, melamine, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, polyurethane, polyacrylate, natural and synthetic elastomers, rubber, acetal, nylon, polyester, styrene polybutadiene, acrylic resin, polyvinylidene chloride, polycarbonate, homopolymers and copolymers of vinyl compounds, polyvinylchloride, polyvinylchloride acetate.

Additional information related to polymers and preparation thereof include PCT Publications No. 98/12243, 98/19713, 98/41154, 99/07417, 00/33764, 00/21842, 00/09190, 00/09088, 00/09087, 2001/17575 and 2001/15526; and U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,099,663, 6,106,473, 6,110,483, 6,121,027, 6,156,345, 6,179,817, 6,197,061, 6,214,901, 6,335,029, and 6,344,035.

Polymers can be fashioned in a variety of forms, with desired release characteristics and/or with specific desired properties. For example, polymers can be fashioned to release a therapeutic agent upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48:343-354, 1993; Dong et al., J. Controlled Release 19:71-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid)-based polymers and derivatives (including, for example, homopolymers such as poly(aminocarboxylic acid), poly(acrylic acid), poly(methyl acrylic acid), copolymers of such homopolymers, and copolymers of poly(acrylic acid) and acrylmonomers such as those discussed above). Other pH sensitive polymers include polysaccharides such as carboxymethyl cellulose, hydroxypropylmethylcellulose phthalate, hydroxypropyl-methylcellulose acetate succinate, cellulose acetate trimellitate, chitosan and alginates. Yet other pH sensitive polymers include any mixture of a pH sensitive polymer and a water soluble polymer.

Polymers also can be employed that are temperature sensitive (see, e.g., Chen et al., “Novel Hydrogels of a Temperature-Sensitive Pluronic Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int′l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, OR, pp. 820-821; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, OR, pp. 829-830; Zhou and Smid, “Physical Hydrogels of Associative Star Polymers,” Polymer Research Institute, Dept. of Chemistry, College of Environmental Science and Forestry, State Univ. of New York, Syracuse, NY, pp. 822-823; Hoffman et al., “Characterizing Pore Sizes and Water ‘Structure’ in Stimuli-Responsive Hydrogels,” Center for Bioengineering, Univ. of Washington, Seattle, WA, p. 828; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int′l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, UT, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermo-gelling polymers include homopolymers such as poly(N-methyl-N-n-propylacrylamide), poly(N-n-propylacrylamide), poly(N-methyl-N-isopropyl-acrylamide), poly(N-n-propylmethacrylamide), poly(N-isopropylacrylamide), poly(N, n-diethylacrylamide), poly(N-isopropylmethacrylamide), poly(N-cyclopropylacrylamide), poly(N-ethylmethyacrylamide), poly(N-methyl-N-ethylacrylamide), poly(N-cyclopropylmethacrylamide) and poly(N-ethylacrylamide); as well as copolymers between (among) monomers of the above list, or by combining such homopolymers with other water soluble polymers such as acrylmonomers (e.g., acrylic acid and derivatives thereof such as methylacrylic acid, acrylate and derivatives thereof such as butyl methacrylate, acrylamide, and N-n-butyl acrylamide). Other representative examples of thermo-gelling cellulose ether derivatives such as hydroxypropyl cellulose, methyl cellulose, hydroxypropylmethyl cellulose, ethylhydroxyethyl cellulose, and PLURONIC® poloxamer synthetic tri-block copolymer, such as F-127.

Therapeutic compositions of embodiments of the present disclosure are fashioned in a manner appropriate to the intended use. Within certain aspects of the present disclosure, the therapeutic composition are biocompatible, and release one or more biologically active agents over a period of hours to several days.

Therapeutic compositions of the present disclosure may also be prepared in a variety of “paste” or gel forms. For example, within one embodiment of the disclosure, therapeutic compositions are provided which are liquid at one temperature (e.g., temperature greater than 37° C.) and solid or semi-solid at another temperature (e.g., ambient body temperature, or any temperature lower than 37° C.).

Also included are polymers, such as PLURONIC® F-127, which are liquid at a low temperature (e.g., 4° C.) and a gel at body temperature (e.g., 37° C.). Such “thermopastes” may be readily made given the disclosure provided herein.

Within yet other aspects of the disclosure, the therapeutic compositions of the present disclosure may be formed as a film. Such films are generally less than 5, 4, 3, 2 or 1 mm thick, for instance less than 0.75 mm or 0.5 mm thick, and or more particularly less than 500 μm. Such films are generally flexible with a good tensile strength (e.g., greater than 50, preferably greater than 100, and more preferably greater than 150 or 200 N/cm²), good adhesive properties (i.e., readily adheres to moist or wet surfaces), and have controlled permeability.

Within certain embodiments of the disclosure, the therapeutic compositions can also comprise additional ingredients such as surfactants (e.g., PLURONICs® such as F-127, L-122, L-92, L-81, and L-61).

Within further aspects of the present disclosure, polymers are provided which are adapted to contain and release a hydrophobic compound, the carrier containing the hydrophobic compound in combination with a carbohydrate, protein or polypeptide. Within certain embodiments, the polymeric carrier contains or comprises regions, pockets or granules of one or more hydrophobic compounds. For example, within one embodiment of the disclosure, hydrophobic compounds may be incorporated within a matrix which contains the hydrophobic compound, followed by incorporation of the matrix within the polymeric carrier. A variety of matrices can be utilized in this regard, including for example, carbohydrates and polysaccharides, such as starch, cellulose, dextran, methylcellulose, and hyaluronic acid, proteins or polypeptides such as albumin, collagen and gelatin. Within alternative embodiments, hydrophobic compounds may be contained within a hydrophobic core, and this core contained within a hydrophilic shell.

Other carriers that may likewise be utilized to contain and deliver the agents described herein include: hydroxypropyl β-cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), implants (Jampel et al., Invest. Ophthalm. Vis. Science 34(11): 3076-3083, 1993; Walter et al., Cancer Res. 54:22017-2212, 1994), nanoparticles (Violante and Lanzafame PAACR), nanoparticles-modified (U.S. Pat. No. 5,145,684), nanoparticles (surface modified) (U.S. Pat. No. 5,399,363), taxol emulsion/solution (U.S. Pat. No. 5,407,683), micelle (surfactant) (U.S. Pat. No. 5,403,858), synthetic phospholipid compounds (U.S. Pat. No. 4,534,899), gas borne dispersion (U.S. Pat. No. 5,301,664), foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), taxoid-based compositions in a surface-active agent (U.S. Pat. No. 5,438,072), liquid emulsions (Tarr et al., Pharm Res. 4:62-165, 1987), nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994) and implants (U.S. Pat. No. 4,882,168).

The agents provided herein can also be formulated as a sterile composition (e.g., by treating the composition with ethylene oxide or by irradiation), packaged with preservatives or other suitable excipients suitable for administration to humans. Similarly, the devices provided herein (e.g., coated catheter) may be sterilized and prepared suitable for implantation into humans.

### IV. Medical Implants

Representative Medical Implants: A wide variety of implants or devices as claimed can be coated with or otherwise constructed to contain and/or release the therapeutic agents provided herein. Though exemplified with CNS implants, it is believed that the advances describe herein will work to minimize cell deposition and/or protein adsorption on other implants in other parts of the body. Representative examples include cardiovascular devices (e.g., implantable venous catheters, venous ports, tunneled venous catheters, chronic infusion lines or ports, including hepatic artery infusion catheters, pacemakers and pacemaker leads (see, e.g., U.S. Pat. Nos. 4,662,382, 4,782,836, 4,856,521, 4,860,751, 5,101,824, 5,261,419, 5,284,491, 6,055,454, 6,370,434, and 6,370,434), implantable defibrillators (see, e.g., U.S. Pat. Nos. 3,614,954, 3,614,955, 4,375,817, 5,314,430, 5,405,363, 5,607,385, 5,697,953, 5,776,165, 6,067,471, 6,169,923, and 6,152,955)); neurologic/neurosurgical devices (e.g., ventricular peritoneal shunts, ventricular atrial shunts, nerve stimulator devices, dural patches and implants to prevent epidural fibrosis post-laminectomy, devices for continuous subarachnoid infusions); gastrointestinal devices (e.g., chronic indwelling catheters, feeding tubes, portosystemic shunts, shunts for ascites, peritoneal implants for drug delivery, peritoneal dialysis catheters, and suspensions or solid implants to prevent surgical adhesion); genitourinary devices (e.g., uterine implants, including intrauterine devices (IUDs) and devices to prevent endometrial hyperplasia, fallopian tubal implants, including reversible sterilization devices, fallopian tubal stents, artificial sphincters and periurethral implants for incontinence, ureteric stents, chronic indwelling catheters, bladder augmentations, or wraps or splints for vasovasostomy, central venous catheters (see, e.g., U.S. Pat. Nos. 3,995,623, 4,072,146 4,096,860, 4,099,628, 4,134,402, 4,180,068, 4,385,631, 4,406,856, 4,568,329, 4,960,409, 5,176,661, 5,916,208), urinary catheters (see, e.g. U.S. Pat. Nos. 2,819,718, 4,227,533, 4,284,459, 4,335,723, 4,701,162, 4,571,241, 4,710,169, and 5,300,022)); prosthetic heart valves (see, e.g., U.S. Pat. Nos. 3,656,185, 4,106,129, 4,892,540, 5,528,023, 5,772,694, 6,096,075, 6,176,877, 6,358,278, and 6,371,983), vascular grafts (see, e.g. U.S. Pat. Nos. 3,096,560, 3,805,301, 3,945,052, 4,140,126, 4,323,525, 4,355,426, 4,475,972, 4,530,113, 4,550,447, 4,562,596, 4,601,718, 4,647,416, 4,878,908, 5,024,671, 5,104,399, 5,116,360, 5,151,105, 5,197,977, 5,282,824, 5,405,379, 5,609,624, 5,693,088, and 5,910,168), ophthalmologic implants (e.g., multino implants and other implants for neovascular glaucoma, drug eluting contact lenses for pterygiums, splints for failed dacrocystalrhinostomy, drug eluting contact lenses for corneal neovascularity, implants for diabetic retinopathy, drug eluting contact lenses for high risk corneal transplants); otolaryngology devices (e.g., ossicular implants, Eustachian tube splints or stents for glue ear or chronic otitis as an alternative to transtempanic drains); plastic surgery implants (e.g., breast implants or chin implants), catheter cuffs and orthopedic implants (e.g., cemented orthopedic prostheses).

Methods of Making Medical Implants having Therapeutic Agents: Implants and other surgical or medical devices may be covered, coated, contacted, combined, loaded, filled, associated with, or otherwise adapted to release (e.g., elute) biologically active agents (e.g., therapeutic agents, such as neutralizing antibodies to and/or other inhibitors of the production or activity of TLR-4, TNF-α, IL-1α, IL-1β, TLR2/6, IL-6, IL-10, or IFN-γ) and compositions of the present disclosure in a variety of manners, including for example: by directly affixing to the implant or device an agent or composition (e.g., by either spraying the implant or device with a polymer/drug film, or by dipping the implant or device into a polymer/drug solution, or by other covalent or noncovalent association means); by coating the implant or device with a substance, such as a hydrogel, that will in turn absorb the composition (or agent); by interweaving agent or composition coated thread into or onto the implant or device; by inserting the implant or device into a sleeve or mesh which is comprised of or coated with a therapeutic composition; constructing the implant or device itself with an agent or composition; or by otherwise adapting the implant or device to release the agent or a composition containing the agent. Within embodiments of the disclosure, the composition should firmly adhere to the implant or device during storage and at the time of insertion. The therapeutic agent or composition should also preferably not degrade during storage, prior to insertion, or when warmed to body temperature after insertion inside the body (if this is required). In addition, it should preferably coat or cover the desired areas of the implant or device smoothly and evenly, with a uniform distribution of therapeutic agent. Within embodiments of the disclosure, the therapeutic agent or composition should provide a uniform, predictable, prolonged release of the therapeutic factor into the tissue surrounding the implant or device once it has been deployed. For vascular stents, in addition to the above properties, the composition should not render the stent thrombogenic (causing blood clots to form), or cause significant turbulence in blood flow (more than the stent itself would be expected to cause if it was uncoated).

Within embodiments of the disclosure, a therapeutic agent can be deposited directly onto all or a portion of the device (see, e.g., U.S. Pat. Nos. 6,096,070 and 6,299,604), or admixed with a delivery system or carrier (e.g., a polymer, liposome, or vitamin as discussed above) that is applied to all or a portion of the device.

Within aspects of the disclosure, biologically active agents and therapeutic agents can be attached to a medical implant using non-covalent attachments. For example, for compounds that are relatively sparingly water soluble or water insoluble, the compound can be dissolved in an organic solvent a specified concentration. The solvent chosen would not result in dissolution or swelling of the polymeric device surface. The medical implant can then be dipped into the solution, withdrawn and then optionally dried (e.g., air dry and/or vacuum dry). Alternatively, a solution of the agent/composition can be sprayed onto the surface of the implant, which can be accomplished using recognized spray coating technology. The release duration for such coating would be relatively short, and would be influenced by the solubility of the agent/composition in the body fluid in which it was placed (e.g., cerebrospinal fluid).

In another aspect, agent(s) can be dissolved in a solvent that has the ability to swell or partially dissolve the surface of a polymeric implant. Depending on the solvent/implant polymer combination, the implant could be dipped into a composition/solution containing the agent for a period of time such that the drug can diffuse into the surface layer of the polymeric device. Alternatively, the agent solution can be sprayed onto all or a part of the surface of the implant, particularly at least part of the surface that will come into contact with a bodily fluid (such as cerebrospinal fluid) after the device is implanted. The release profile of the agent depends upon the solubility of the agent in the surface polymeric layer. Using this approach, the solvent is selected so it does not result in a significant distortion or dimensional change of the medical implant.

If the implant is composed of material(s) that do not allow incorporation of a therapeutic agent into the surface layer using a solvent method, the surface of the device can be treated with a plasma polymerization method such that a thin polymeric layer is deposited onto the device surface. Examples of such methods include parylene coating of devices, and the use of monomers such hydrocyclosiloxane monomers, acrylic acid, acrylate monomers, methacrylic acid or methacrylate monomers. Dip coating or spray coating methods described above can then be used to incorporate the therapeutic agent into the coated surface of the implant.

For biologically active agents that have some degree of water solubility, the retention of these compounds on a device are relatively short-term. For therapeutic agents that contain ionic groups, it is possible to ionically complex these agents to oppositely charged compounds that have a hydrophobic component. For example therapeutic agents containing amine groups can be complexed with compounds such as sodium dodecyl sulfate (SDS). Compounds containing carboxylic groups can be complexed with tridodecymethyammonium chloride (TDMAC). Mitoxantrone, for example, has two secondary amine groups and comes as a chloride salt. This compound can be added to sodium dodecyl sulfate in order to form a complex. This complex can be dissolved in an organic solvent which can then be dip coated or spray coated.

For agents that have the ability to form ionic complexes or hydrogen bonds, the release of these agents from the device can be modified by the use of organic compounds that have the ability to form ionic or hydrogen bonds with the therapeutic agent. A complex between the ionically charged therapeutic agent and an oppositely charged hydrophobic compound can be prepared prior to application of this complex to the medical implant. In another embodiment, a compound that has the ability to form ionic or hydrogen bond interactions with the therapeutic agent can be incorporated into the implant during the manufacture process, or during the coating process. Alternatively, this compound can be incorporated into a coating polymer that is applied to the implant or during the process of loading the therapeutic agent into or onto the implant. These agents can include fatty acids (e.g., palmitic acid, stearic acid, lauric acid), aliphatic acids, aromatic acids (e.g., benzoic acid, salicylic acid), cylcoaliphatic acids, aliphatic (stearyl alcohol, lauryl alcohol, cetyl alcohol) and aromatic alcohols alco multifunctional alcohols (e.g., citric acid, tartaric acid, pentaerithratol), lipids (e.g., phosphatidyl choline, phosphatidylethanolamine), carbohydrates, sugars, spermine, spermidine, aliphatic and aromatic amines, natural and synthetic amino acids, peptides or proteins.

Agents containing amine groups can form ionic complexes with sulfonic or carboxylic pendant groups or end-groups of a polymer. Examples of polymers that can be used for this application include, but are not limited to polymers and copolymers that are prepared using acrylic acid, methacrylic acid, sodium styrene sulfonate, styrene sulfonic acid, maleic acid or 2-acrylamido-2-methyl propane sulfonic acid. Polymers that have been modified by sulfonation post-polymerization can also be used in this application. The medical implant, for example, can be coated with, or prepared with, a polymer that comprises NAFION™ (a sulfonated fluoropolymer). This medical device can then be dipped into a solution that comprises the amine-containing therapeutic agent. The amine-containing therapeutic agent can also be applied by a spray coating process.

Agents with available functional groups can be covalently attached to the medical implant surface using several chemical methods. If the polymeric material used to manufacture the implant has available surface functional groups then these can be used for covalent attachment of the agent. For example, if the implant surface contains carboxylic acid groups, these groups can be converted to activated carboxylic acid groups (e.g. acid chlorides, succinimidyl derivatives, 4-nitrophenyl ester derivatives, and so forth). These activated carboxylic acid groups can then be reacted with amine functional groups that are present on the therapeutic agent (e.g., methotrexate, mitoxantrone).

For surfaces that do not contain appropriate functional groups, such groups can be introduced to the polymer surface via a plasma treatment regime. For example, carboxylic acid groups can be introduced via a plasma treatment process (e.g., the use of O₂ and/or CO₂ as a component in the feed gas mixture). The carboxylic acid groups can also be introduced using acrylic acid or methacrylic acid in the gas stream. These carboxylic acid groups can then be converted to activated carboxylic acid groups (e.g., acid chlorides, succinimidyl derivatives, 4-nitrophenyl ester derivatives, etc.) that can subsequently be reacted with amine functional groups that are present on agent(s).

In addition to direct covalent bonding to an implant surface, agents with available functional groups can be covalently attached to the medical implant via a linker. Such linkers can be degradable or non-degradable. Linkers that are hydrolytically or enzymatically cleaved are appropriate in various embodiments. These linkers can comprise azo, ester, amide, thioester, anhydride, or phosphor-ester bonds.

To further modulate the release of the therapeutic agent from the medical implant, portions of or the entire medical implant may be further coated with a polymer. The polymer coating can comprise polymer(s) such as those described above. The polymer coating can be applied by a dip coating process, a spray coating process, or a plasma deposition process, for instance. This coating can, if desired, be further crosslinked using thermal, chemical, or radiation (e.g., visible light, ultraviolet light, e-beam, gamma radiation, x-ray radiation) techniques in order to further modulate the release of the therapeutic agent from the medical implant. This polymer coating can further contain agents that can increase the flexibility (e.g., plasticizer-glycerol, triethyl citrate), lubricity (e.g., hyaluronic acid), biocompatibility or hemocompatibility (e.g., heparin) of the coating.

The methods above describe methods for incorporation of a therapeutic agent into or onto a medical implant. Other agents may also be included on the medical implant. For instance, antibacterial or antifungal agents can also be incorporated into or onto the medical implant. Such antibacterial or antifungal agents can be incorporated into or onto the medical implant prior to, simultaneously or after the incorporation of the biologically active agents, described above, into or onto the medical implant. Agents that can be used include, but are not limited to silver compounds (e.g., silver chloride, silver nitrate, silver oxide), silver ions, silver particles, iodine, povidone/iodine, chlorhexidine, 2-p-sulfanilyanilinoethanol, 4,4′-sulfinyldianiline, 4-sulfanilamidosalicylic acid, acediasulfone, acetosulfone, amikacin, amifloxacin, amoxicillin, amphotericin B, ampicillin, apalcillin, apicycline, apramycin, arbekacin, aspoxicillin, azaserine, azidamfenicol, azithromycin, aztreonam, bacitracin, bambermycin(s), biapenem, brodimoprim, butirosin, candicidin(s), capreomycin, carbenicillin, carbomycin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlorphenesin, chlortetracycline, ciprofloxacin, clarithromycin, clinafloxacin, clindamycin, clomocycline, colistin, cyclacillin, dapsone, demeclocycline, dermostatin(s), diathymosulfone, dibekacin, dihydrostreptomycin, dirithromycin, doxycycline, enoxacin, enviomycin, epicillin, erythromycin, filipin, fleroxacin, flomoxef, fortimicin(s), fungichromin, gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, mepartricin, meropenem, methacycline, micronomicin, midecamycin(s), minocycline, moxalactam, mupirocin, nadifloxacin, natamycin, neomycin, netilmicin, norfloxacin, nystatin, ofloxacin, oleandomycin, oligomycin(s), oxytetracycline, p-sulfanilylbenzylamine, panipenem, paromomycin, pazufloxacin, pefloxacin, penicillin N, perimycin A, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, rosoxacin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoldine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin, tubercidin, vancomycin, and the like.

See also teachings in WO 03/099346.

### V. Clinical Applications

In order to further the understanding of the disclosure, discussed below are representative clinical applications for the compositions, methods, and devices provided herein.

Briefly, as noted above, within one aspect of the disclosure modified implants are provided for preventing, reducing, and/or inhibiting cell deposition on (and/or the attendant blockage and failure of) a medical implant. Such modified implants include or contain (e.g., are coated or partially coated with) a biologically active agent or composition as described herein. A method of using the modified implants comprises introducing into a patient a medical implant that releases a biologically active agent, wherein the biologically active agent reduces, inhibits, or prevents deposition of cells (such as astrocytes and/or glia cells) on the surface of a medical implant device. As used herein, agents that reduce, inhibit, or prevent such cellular deposition in a patient means that the cellular deposition (and/or associated device blockage or failure) is reduced, inhibited, or prevented in a statistically significant manner in at least one clinical outcome, or by any measure routinely used by persons of ordinary skill in the art as a diagnostic criterion in determining the same. In a representative embodiment, the medical implant has been covered or coated with a neutralizing antibody specific for TLR-4, TNF-α, IL-1α, or IL-1β, or more particularly neutralizing antibodies for one or more of TNF-α, IL-1β, or IL-6; or an inhibitor of production or activity of TLR-4, TNF-α, IL-1α, IL-1β, IL-6, TLR2/6, IL-10, or IFN-γ. In particular embodiments, one or more inhibitors of a component involved with stimulation of A2 reactive astrocytes are used.

It is particularly contemplated that representative implant devices provided herein are central nervous system implants, such as a ventricular catheter or another component of a system used to treat or manage hydrocephalus. Optionally, the ventricular catheter is formatted for use with a hydrocephalus shunt.

Drug elution/drug coating with a tuned elution/activity range for maximal effect when cytokines are the highest would all be done in advance to the proximal catheter of the shunt system. Essentially, there need be no difference for the clinician—which provides a major advantage of the advances provided herein. In fact, the clinical may not even notice the addition of the herein described active agent(s), for instances if it is provided in a coating/impregnated into the shunt.

In limited embodiments, the clinical administration system may include or involve an osmotic pump that is used to drive the drug delivery, this would be similar in technology to the ReFlow system (Anuncia), where they backflush saline into the ventricular system—one or more of the herein-described active agent(s) may be added to the backflush, for instance. Optionally, this is done in a controlled rate.

It is envisioned in some embodiments provide a catheter impregnated with GIT27, which can be used in a clinical setting without significant changes for the clinician.

In some embodiments, if the anti-inflammatory active agent is to be added “pre-treatment” (that is, before insertion or implantation of a medical implant), the clinical procedure may include intraperitoneal (IP) injection of a composition containing the active agent(s).

### VI. Shunt Systems Comprising a Catheter

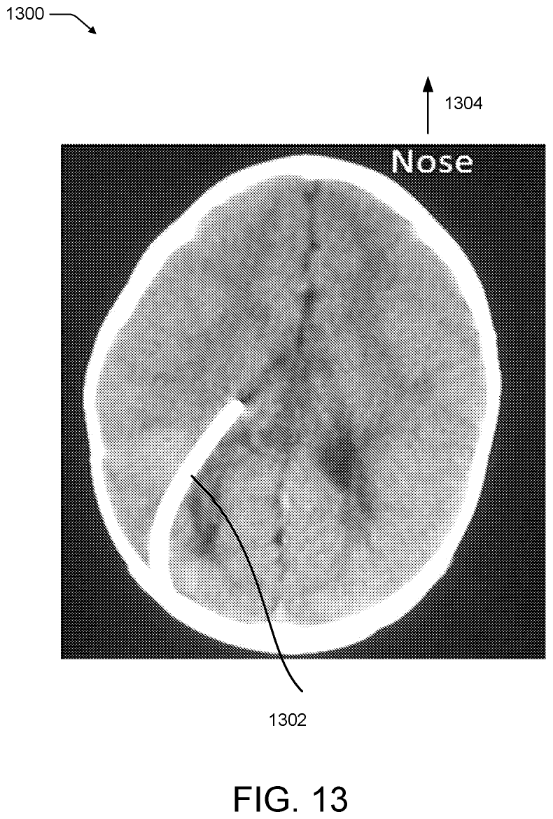

As described herein, the common treatment for hydrocephalus patients is CSF drainage by shunting. A shunt system that employs the cell deposition reduction treatment(s) described herein (e.g., where an implant component of the shunt system contains or is coated with at least one neutralizing antibody specific for one of TLR-4, TNF-α, IL-1α, IL-1β, IL-6, TLR2/6, IL-10, or IFN-γ, or at least one inhibitor of production or activity of one of TLR-4, TNF-α, IL-1α, IL-1β, IL-6, TLR2/6, IL-10, or IFN-γ) can be implemented into a patient's brain by surgical insertion to treat hydrocephalous patients. FIG. 13 illustrates a horizontal head CT image 1300 showing a catheter tip implanted in a patient's brain. Arrow 1304 shows the direction of the patient's nose. Referring to FIG. 13, the image 1300 shows a catheter tip 1302 surrounded by tissue. While many factors such as infection and disconnection could lead to shunt obstruction and eventual failure, the statistics tell us that most shunts fail by becoming blocked with cells and tissues. As described above, shunt geometry with a more uniform flow rate distribution among the shunt's inlet holes would reduce the obstruction occurring at the critical proximal inlet holes, thereby reducing shunt failure rates.

This disclosure further provides a shunt system comprising the catheter for shunting biological fluid flow as described above. Such a shunt system offers a more uniform flow rate distribution among the inlet holes of the catheter, and would reduce the obstruction occurring at the inlet holes, thereby reducing shunt failure rates. The shunt system is can improve patients' quality of life by reducing the shunt failure rate. In some instances, the system can also be used to conduct in vitro experiments, collect analytic data, validate shunt functions, etc.

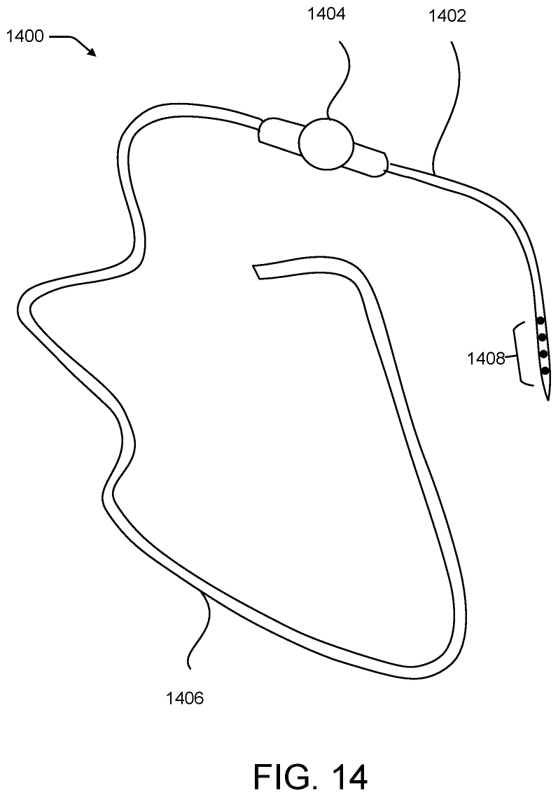

FIG. 14 illustrates an example shunt system 1400 in accordance with implementations of this disclosure. In some examples, the shunt system 1400 can be used in vitro to study fluid flow, or in vivo to treat or ameliorate symptoms of hydrocephalus. Referring to FIG. 14, the shunt system 1400 includes a ventricle catheter 1402, a valve 1404, and a distal catheter 1406. The valve 1404 is connected between the ventricular catheter 1402 and the distal catheter 1406. In some examples, the shunt system 1400 can be made of biologically compatible material such as polydimethylsiloxane (PDMS, silicone) or the like.

The ventricle catheter 1402 can be implemented into a patient's brain ventricle. The ventricle catheter 1402 includes multiple holes 508 that allow biological fluid flow through into the ventricle catheter 1402.

The valve 1404 is configured to regulate the biological fluid (such as CSF) flowing therethrough. In some examples, the valve 1404 can be opened and closed. In some examples, the valve 1404 can regulate the flow rate of the biological fluid. In some examples, the valve 1404 can be a conventional valve. In some examples, the valve 1404 can optionally be a solid state valve.

The distal catheter 1406 is configured to introduce the biological fluid to another part of the body, such as the abdomen through the peritoneum of the patient. As such, excess CSF of the hydrocephalous patient can be drained from the brain to another part of the body where CSF can be more easily absorbed.

Example shunts are composed of two polydimethylsiloxane (PDMS, silicone) shunt catheters connected by a pressure valve. One catheter remains in the ventricles, while the other is tunneled subcutaneously into the peritoneum or atrium.

### VII. Kits

Also provided are kits useful for treating hydrocephalus patients. An example of the kit includes one or more of: a ventricular catheter that contains or has been treated with a biologically active agent as described herein (e.g., at least one neutralizing antibody specific for one of TLR-4, TNF-α, IL-1α, IL-1β, IL-6, TLR2/6, IL-10, or IFN-γ, or at least one inhibitor of production or activity of one of TLR-4, TNF-α, IL-1α, IL-1β, IL-6, TLR2/6, IL-10, or IFN-γ), a shunt valve, and a distal catheter, each of which is sterile, and vacuum sealed.

More generally, kits can include instructions, for example, written instructions, on how to use the material(s) therein. Material(s) can be, for example, any substance, composition, solution, etc., herein or in any patent, patent application publication, reference, or article that is incorporated by reference.

A kit can include a shunt system as described herein, and optionally additional components such as buffers, reagents, and instructions for carrying out the methods described herein. The choice of buffers and reagents will depend on the particular application, e.g., setting of the assay (point-of-care, research, clinical), analyte(s) to be assayed, the detection moiety used, the detection system used, etc.

The kit can also include informational material, which can be descriptive, instructional, marketing, or other material that relates to the methods described herein and/or the use of the devices for the methods described herein. In embodiments, the informational material can include information about the production of the device, physical properties of the device, date of expiration, batch or production site information, and so forth.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### VIII. Exemplary Embodiments

1. An implantable medical device including: a coating including a biologically active agent over at least a portion of the surface of the device that is exposed to a biological fluid when the device is implanted, wherein the biologically active agent includes: a neutralizing antibody specific for TLR-4, IL-1β, IL-6, TNF-α, IL-1α, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

2. A medical implant that releases a biologically active agent in an amount effective to reduce or inhibit astrocyte and/or glia cell deposition associated with the medical implant, wherein the biologically active agent is selected from the group consisting of: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ, and wherein the medical implant includes 0.1 μg to 1 mg of biologically active agent per mm² of surface area of the portion of the medical implant to which the biologically active agent is applied or incorporated.

3. The medical implant of embodiment 2, which includes one or more of a tube, a chronic indwelling central nervous system (CNS) catheter, a neurological or neurosurgical device, a central nervous shunt, a pump, or a catheter.

4. A ventricular shunt coated with GIT 27 or another TLR-4 inhibitor.

5. An improved shunt catheter system, the improvement including in the system at least one biologically active agent selected from the group consisting of: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

6. The improved shunt catheter system of embodiment 6, wherein the inhibitor of production or activity of TNF-α includes one or more of: a neutralizing antibody specific for TNF-α, etanercept, infliximab, adalimumab, certolizumab pegol, or golimumab.

7. The improved shunt catheter system of embodiment 6, wherein the inhibitor of production or activity of IL-1α or IL-13 includes one or more of: a neutralizing antibody for IL-1, a neutralizing antibody specific for IL-1α, a neutralizing antibody specific for IL-1β, anakinra, Rilonacept, or canakinumab.

8. The improved shunt catheter system of embodiment 6, wherein the inhibitor of production or activity of TLR-4 includes one or more of: a neutralizing antibody of TLR-4, GIT 27, Eritoran, ibudilast, NI-0101, 1A6, or 15C1.

9. The improved shunt catheter system of embodiment 6, wherein the inhibitor of production of IL-6 includes one or more of Tocilizumab, Sarilumab, Clazakizumab, Olokizumab, ALX-006,

10. A method of reducing cellular deposition and/or shunt device blockage in a fluid moving system, including introducing into the system: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

11. A method of reducing shunt device failure, including: inhibiting astrocyte and/or glia cell activation by contacting the shunt and/or a fluid passing over/through the shunt with: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

12. A method of mitigating astrocyte immune response to a chronically indwelling neuroprosthetic device, including: inhibiting secretion and/or activity of at least one of TNF-α, IL-1β, or IL-6; or inhibiting secretion and/or activity of TLR-4, TLR2/6, or IFN-γ.

13. The method of any one of embodiments 9-12, wherein the method inhibits formation or reverses formation of glial scar.

14. The method of any one of embodiments 9-12, wherein the method inhibits obstruction of one or more openings in the device.

15. A method of reducing astrocyte activation and attachment on a surface of a shunt, including contacting the shunt surface with a composition including: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

16. A method of reducing obstruction/blockage and failure of a ventricular shunt in a hydrocephalus patient, including contacting the shunt, before or after installation of the shunt in the patient, with a composition including: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

17. A method to decrease cellular attachment to a catheter surface, including contacting a surface of the catheter with a composition including: a neutralizing antibody specific for TLR-4, TNF-α, IL-1α, IL-1β, IL-6, TLR2/6, IL-10, or IFN-γ; an inhibitor of production or activity of TLR-4, TNF-α, IL-6, or IL-1β; or an inhibitor of production or activity of TLR2/6, IL-10, IL-1α, or IFN-γ.

18. A method of decreasing astrocyte cell attachment on a medical implant device, including contacting the device with GIT 27 or another TLR-4 inhibitor before installation of the medical implant device.

19. A method of decreasing secretion of cytokines from astrocyte cells in a neurological implant system, including contact the astrocyte cells with GIT 27 or another TLR-4 inhibitor during or after installation of a neurological implant device.

20. A device, method, system, or composition for inhibiting or reducing cellular deposition on a medical implant, essentially as described herein.

### IX. Experimental Examples

### Example 1: The Effect of A1/A2 Reactive Astrocyte Expression on Hydrocephalus Shunt Failure

Understanding the composition characteristics of the glial scar contributing to the high failure rate of neuroprosthetic devices implanted in the brain has been limited, to date, with the evaluation of cells, tissue, and biomarkers obstructing the implant. However, there remains a critical knowledge gap in gene expression profiles of the obstructing cells. This example investigates the phenotypic expression specific to astrocyte scarring from those cells on hydrocephalus shunt surfaces at the time of failure, aimed at the development of therapeutic approaches to target reactive astrocytes for improved functional outcome. Recent evidence has indicated that the tissue obstructing shunts is over 80% inflammatory, with a more exaggerated astrocytic response.

To understand how to mitigate the astrocyte immune response to shunts, gene expression profiling of the C3 and EMP1 genes was performed to quantify if astrocytes were classically activated and pro-inflammatory (A1) or alternatively activated and anti-inflammatory (A2), respectively. Shunt catheters were removed from patients at the time of failure and categorized by obstructed vs non-obstructed shunts. RNAscope fluorescent in situ hybridization and quantitative PCR analysis of the C3 and EMP1 expressed genes revealed that a heterogeneous mixed population of both the A1 and A2 reactive phenotype exist on the shunt surface. However, the number of A2 reactive astrocytes is significantly higher on obstructed shunts compared to A1 reactive astrocytes. ELISA data also confirmed higher levels of IL-6 for obstructed shunts involved in A2 reactive astrocyte proliferation and glial scar formation on the shunt surface.

Since TNF-α and IL-1β propel resting astrocytes into an A2 reactive state, by simply blocking the secretion or action of these cytokines, astrocyte activation and attachment on obstructing shunts could be inhibited. Specifically, this Example provides evidence that it is effective and beneficial to inhibit one or more of the cytokines involved in activating A2 reactive astrocytes. At least some of the information included in this Example was published online Nov. 4, 2021 as Khodadadei et al., at biorxiv.org/content/10.1101/2021.11.04.467357v1.

Implantation of foreign materials within the brain initiates a series of reactions, collectively called the foreign body reaction (FBR), which aims to eliminate or isolate the implanted foreign material from the host immune system. Upon implantation of large medical devices such as neuroprosthetics, where elimination is not possible, the FBR continues until the device is barricaded from healthy brain tissue. The initial phase of the FBR is blood-device interactions, which occurs immediately upon implantation caused by vasculature or blood-brain barrier (BBB) disruption. This results in the nonspecific adsorption of blood proteins to the device surface through a thermodynamically driven process to reduce surface energy. Other than BBB disruption and influx of serum proteins, the immune system is also activated by signals of host cell injury and extracellular matrix (ECM) breakdown proteins such as fibrinogen and fibronectin adhesion to the device surface. Microglia, the resident immune cells of the central nervous system (CNS), and blood-derived macrophages recognize the protein signals through receptor-mediated pathways such as toll like receptors (TLRs). Ligand binding to TLRs leads to activation of microglia/macrophages and the secretion of pro-inflammatory cytokines such as TNF-α, IL-1α, and IL-1B (Bedell et al., Acta Biomater. 102:205-219, 2020; Hermann et al., Front. Bioeng. Biotechnol. 6:1-17, 2018). These very potent signaling molecules are rapidly upregulated in the injured CNS, and are observed right at the device-tissue interface corresponding to the location of activated microglia/macrophages and exaggerated astrocytes (Hermann et al., Front. Bioeng. Biotechnol. 6:1-17, 2018; Karumbaiah et al., Biomaterials. 34(33):8061-8074, 2013; Tomaszewski, J Neural Eng. 2015; 12(1):11001; Shinozaki et al., Cell Rep. 19(6)1151-1164, 2017; Jorfi et al., J Neural Eng. 12(1), 2015). The effect of TNF-α and IL-1β is strongest on astrocyte activation and proliferation, the key member of the CNS immune response. Reactive astrocytes form a physical barrier, known as glial scar, where newly formed and hypertrophic astrocytes overlap and play a beneficial role to prevent injury from spreading to surrounding healthy tissue. However, in relation to its effect on implants, the glial scar is considered undesirable because, regardless of the device type, it elicits failure (Moshayedi et al., Biomaterials. 35(13):3919-3925, 2014; He & Bellamkonda, PMID: 201204399. Boca Raton (FL): CRC Press/Taylor & Francis; 2008. Chapter 6). Collectively, the dominant role of cytokines in orchestrating the dynamic crosstalk among cells and mediating device failure is evident.

A deeper understanding of astrocyte phenotype leads to a more accurate interpretation of failure in chronically indwelling neuroprosthetics. In a recent world-renowned study, Barres and colleagues revealed two significantly different reactive astrocyte phenotype, A1 and A2 (Liddelow et al., Nature. 541(7638):481-487, 2017; Liddelow & Barres, Immunity. 46(6):957-967, 2017). The A1 reactive astrocytes produce large volumes of pro-inflammatory substances and neurotoxin that can induce neuronal death. The A2 reactive astrocytes upregulate anti-inflammatory substances and many neurotrophic factors, which promote survival and growth of neurons. The A1 neuroinflammatory astrocytes are induced by NF-κB signaling, whereas the A2 scar-forming, proliferative astrocytes are induced by STAT3-mediated signaling (Liddelow et al., Nature. 541(7638):481-487, 2017; Zamanian et al., J Neurosci. 32(18):6391-6410, 2012). Since glial scar borders are formed by newly proliferated, elongated astrocytes via STAT3-dependent methods, studies strongly suggest that the A2 reactive astrocyte phenotype is present during glial scar formation (Liddelow & Barres, Immunity. 46(6):957-967, 2017; Anderson et al., Nature. 532(7598):195-200, 2016; Wanner et al., J Neurosci. 2013; 33(31):12870-12886). Furthermore, in vivo quiescent astrocytes that contact serum upon injury and BBB disruption express many of the A2 reactive astrocyte genes (Zamanian et al., J Neurosci. 32(18):6391-6410, 2012; Zhang et al., Neuron. 89(1):37-53, 2016; Liddelow, FASEB J. 33(2)1528-1535, 2019).

In the brain, TNF-α, IL-1α and C1q combined propel resting astrocytes into an A1 reactive state (Liddelow et al., Nature. 541(7638):481-487, 2017). Co-stimulation with TNF-α and IL-1β induces the A2 reactive state with neurosupportive characteristics (Hyvarinen et al., Sci Rep. 9(1):1-15, 2019). In fact, TNF-α and IL-1β modulate the glial scar process by stimulating astrocyte IL-6 secretion (Selmaj et al., J Immunol. 144(1):129-135, 1990). IL-6 primarily activates astrocyte proliferation by a positive feed-forward loop, further activating local astrocytes to maintain the glial scar through self-sustaining mechanisms. IL-6 signaling pathways are enhanced in A2 reactive astrocytes, and STAT3 is activated by IL-6 (Zamanian et al., J Neurosci. 32(18):6391-6410, 2012; Lutz et al., Astrocytes Wiring the Brain. 283-310, 2011). IL-6 is one of the initial triggers of reactive astrocytes in the acute phase of disease, involved in improving neuronal survival and neurite growth (Moshayedi et al., Biomaterials. 35(13):3919-3925, 2014; He & Bellamkonda, PMID: 201204399. Boca Raton (FL): CRC Press/Taylor & Francis; 2008. Chapter 6). Although these properties are evidence of the beneficial roles of IL-6 in repair and modulation of inflammation in the CNS, the overproduction of IL-6 is associated with glial scar formation. Hence, a careful inflammatory balance of IL-6 is essential for proper repair. Inhibition of both IL-6 and IL-6r by antibody neutralization has been shown to reduce glial scar formation on the implanted device and damage to the brain as a result of bystander effects of increased CSF cytokine levels (Ramesh et al., Mediators Inflamm. 2013:480739, 2013; doi: 10.1155/2013/480739).

Hydrocephalus is a devastating and costly disease. The most common treatment paradigm is surgical shunting of cerebrospinal fluid (CSF). However, shunts are plagued by unacceptably high failure rates (40% in the first year, 90% in the first ten years) (Vinchon et al., Fluids Barriers CNS. 9(1):1-10, 2012; Vinchon et al., Childs Nerv Syst. 28(6):847-854, 2012; Stone et al., J Neurosurg Pediatr. 11(1)15-19, 2013; Harris & McAllister, Neurosurgery. 70(6):1589-1601, 2012), and impose a significant burden on patients, their families, and society. Understanding the root causes of shunt failure to design improved devices, will indeed reduce these burdens.

Shunts primarily fail due to obstruction of the shunt system with adherent inflammatory cells (Drake et al., Childs Nerv Syst. 16(10-11):800-804, 2000; Browd et al., Pediatr Neurol. 34(2):83-92, 2006; Kestle et al., Pediatr Neurosurg. 84113:230-236, 2001; Pujari, J Neurol Neurosurg Psychiatry. 79(11):1282-1286, 2008; Harris et al., Fluids Barriers CNS. 1-15, 2015; Hanak et al., J Neurosurg Pediatr. 18(2):213-223, 2016). Astrocytes and macrophages are the dominant cell types bound directly to the catheter. Recent work indicates that astrocytes make up more than 21% of cells bound to obstructed shunts. Of the obstructed masses blocking ventricular catheter holes, astrocytes makeup a vast majority of cells. Astrocyte markers in obstructive masses are observed to be co-localized with proliferative markers, indicating that astrocytes are active on the shunt surface; they produce inflammatory cytokine IL-6 and proliferate (Khodadadei et al., Commun Biol. 4(1):1-10, 2021), and their number and reactivity peak on failed shunts. In hydrocephalus patients, IL-6 cytokines significantly increase during shunt failure, especially after repeat failures. Astrocytes create a “glue” for more glia or other cells and tissues to secondarily bind and block the shunt. Even contact with the ventricular wall results in astrocyte migration to the surface and interaction with the shunt (Del Bigio & Bruni, J Neurosurg. 69(1):115-120, 1988).

In this study, a goal is to reduce shunt obstruction by re-developing a strategy to reduce astrocyte activation and thus attachment and density on the shunt surface. Thus, it can be beneficial to control the degree of inflammatory cell activation. This is not the direct target of a specific treatment paradigm to date.

Astrocyte phenotype expression in tissue that is obstructing shunts is first determined, using failed patient's shunts, then a promising pharmacological agent is employed that will inhibit the cell activation state to reduce attachment. The A1 reactive astrocytes are not as quickly proliferative, however, considerable proliferation of A2 reactive astrocytes is seen when the reactive response is to produce a protective scar around the injury (Liddelow & Barres, Immunity. 46(6):957-967, 2017). A goal is to observe whether the cells blocking shunts are expressing an A1 or A2 reactive astrocyte phenotype, which will help to understand how to mitigate the cell immune response to shunts—that is, to reduce mechanisms leading to shunt failure through inhibition of cell activation.

To address this new research avenue, RNAscope fluorescent in situ hybridization and quantitative PCR analysis were used to determine the A1 or A2 reactive astrocyte phenotype expression on failed shunt. ELISA analysis confirmed the pro- and anti-inflammatory cytokine concentration profiles in the CSF associated with astrocyte activation. A powerful marker for A1 is the classical complement cascade component C3, specifically upregulated in A1 reactive astrocytes (and not in resting or A2 reactive astrocytes). C3 is one of the most characteristic and highly upregulated genes in A1 and EMP1 is an A2-specific gene.

The release of a pharmacological agent (such as an FDA-approved pharmacological agent) on the shunt surface can then be employed, that will inhibit the cell activation state to reduce the presence of astrocytes on shunts. This will keep any attaching astrocytes in a resting state, reduce proliferation, inhibit downstream proliferation, and ultimately deter shunt obstruction. Since the master cytokine IL-1 (α and β) is the initial molecular mediator that triggers glial scar formation around other devices in the brain, whether astrocytes obstructing shunts could be prevented by blocking secretion or action of these cytokines to keep astrocytes out of the A1 or A2 reactive state has been investigated. FDA-approved drugs targeting TNF-α, IL-1α and IL-1β already exist and are in use for other medical conditions.

### Materials and Methods

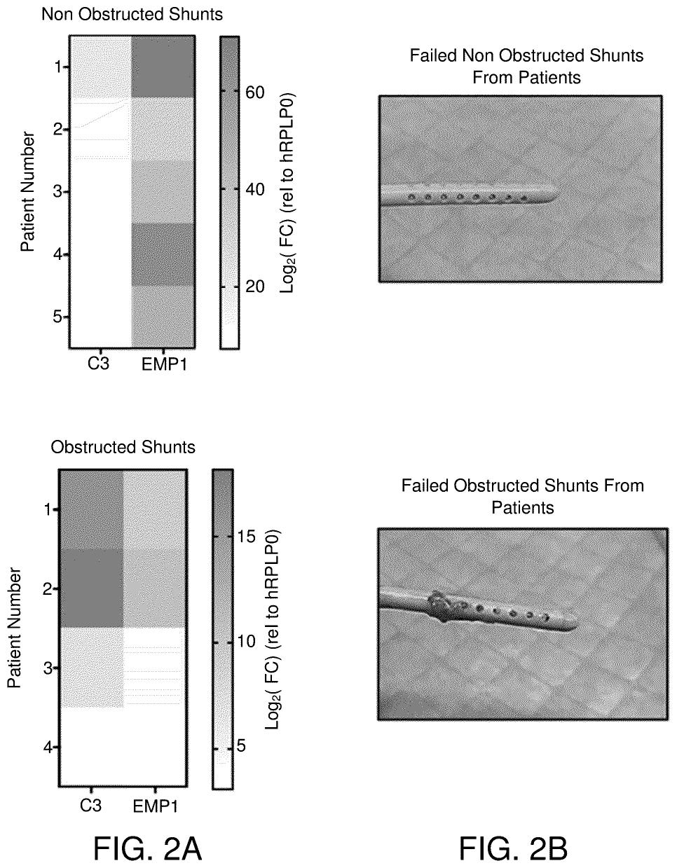

Ethics approval: The permission to collect shunt hardware, CSF, and patient data was approved by the Wayne State Institutional Review Board (IRB) as the coordinating center and as a participating site. Written informed consent was obtained from all patients or their legally authorized representative. Collection was performed in a manner consistent with the standard of treatment; decision to remove the shunt was always based on clinical symptoms for surgical intervention chosen by the neurosurgeon. Samples were collected from individuals with any hydrocephalus etiology and clinical history. After removal by a surgeon, the shunt samples were immediately submerged in a solution of paraformaldehyde (PFA) to fix cells for RNAscope fluorescent in situ hybridization experiments or RNAlater, an aqueous, non-toxic tissue storage reagent that rapidly permeates tissue to stabilize and protect the quality/quantity of cellular RNA in situ in unfrozen specimens for qPCR experiments. RNAlater eliminates the need to immediately process tissue specimens or to freeze samples in liquid nitrogen for later processing. Obstructed and non-obstructed shunts were characterized based on the degree of actual tissue blockage on the shunt surface shown in FIG. 2B. A whole-mount procedure was used for all non-obstructed shunts, where the whole shunt was immersed in solution. For all obstructed shunts the tissue on the surface of the shunt was removed from the shunt. Tissue was then embedded in OCT compound for RNAscope fluorescent in situhybridization experiments or immersed in solution for qPCR experiments. As previously described (Harris et al., Fluids Barriers CNS. 18(1):1-14, 2021), CSF was collected at the time of shunt surgery and transported on ice to the Washington University Neonatal CSF Repository. Samples were then centrifuged (2500 rpm for 6 min) at room temperature, and the supernatant was aliquoted and stored in 1.5 ml polypropylene microcentrifuge tubes at −80° C. until experimental analysis.

Quantitative PCR: Total RNA was extracted using the GenElute Mammalian Total RNA Miniprep Kit (sigma), cDNA synthesis was performed using the iScript cDNA Synthesis Kit (Bio-Rad), and qPCR was completed using the PowerUp SYBR Green Master Mix (Applied Biosystems) according to manufacturer protocols. Relative mRNA expression was normalized to hRPLPO (reference gene) (Guttenplan et al., Nat Commun. 11(1):1-9, 2020). Primers for human are as follows: hC3 (A1 reactive astrocyte marker), hEMP1 (A2 reactive astrocyte marker) (Clarke et al., Proc Natl Acad Sci USA. 115(8):E1896-E1905, 2018).

RNAscope fluorescent in situ hybridization: RNAscope fluorescent in situ hybridization was performed on fixed frozen tissue. Tissue was embedded in OCT compound (Tissue-Tek) and 14 μm tissue sections were prepared and immediately frozen at −80° C. Multiplex RNAscope was performed according to manufacturer's (ACD: Advanced Cell Diagnostics) protocol against the target mRNA probes of hC3 (label for A1 reactive astrocytes), hEMP1 (label for A2 reactive astrocytes), and hSLC1A3 (label for astrocytes). RNAscope fluorescent in situ hybridization is nonlinearly amplified and thus intensity cannot be used to measure expression. Instead, images were thresholded in ImageJ. The percent of area covered by this thresholded signal was then quantified and recorded as reactivity (Guttenplan et al., Nat Commun. 11(1):1-9, 2020). Images were acquired with a resonance-scanning confocal microscopy (RS-G4 upright microscope, Caliber ID, Andover, MA, USA).

Multiplex ELISA: Multiplex assays were run by the Bursky Center for Human Immunology & Immunotherapy Programs (CHiiPs) at Washington University School of Medicine. Frozen supernatant CSF was slowly thawed and then analyzed in duplicate with multiplex immunoassay kits according to the manufacturer's instructions for the following inflammatory cytokines: IL-1α, IL-1β, IL-6, TNF-α, IL-8, IL-4, IL-10 (ThermoFisher Scientific), C3, and C1q (Millipore Sigma). Briefly, magnetic beads were added across all the wells on the plate, CSF samples and standards were then added in duplicate. Following washing steps, the detection antibody was added followed by streptavidin incubation. Beads were then resuspended with reading buffer and data were acquired on a Luminex detection system. The concentration of each analyte was calculated by plotting the expected concentration of the standards against the multiplex fluorescent immunoassay generated by each standard. A 4-parameter logistic regression was used for the best fit curve. Protein concentration is reported as pg/mL for each analyte.

Purification of astrocytes by immunopanning: Astrocytes were purified by immunopanning from post-natal day (P) 5 mouse brains and cultured as previously described (Foo et al., Neuron. 71(5):799-811, 2011). Cerebral cortices were dissected and enzymatically digested using papain at 37° C. and 10% CO₂. Tissue was then mechanically triturated with a serological pipette at RT to generate a single-cell suspension. The suspension was filtered and negatively panned for microglia/macrophage cells (CD45), oligodendrocyte progenitor cells (04 hybridoma), and endothelial cells (L1) followed by positive panning for astrocyte cells (ITGB5). Astrocytes were cultured in defined, serum-free medium containing 50% neurobasal, 50% DMEM, 100 U/mL penicillin, 100 μg/mL streptomycin, 1 mM sodium pyruvate, 292 μg/mL L-glutamine, 1×SATO, 5 μg/mL of N-acetyl cysteine, and 5 ng/mL HBEGF.

All animal protocols were approved by the Institutional Animal Care and Use Committee at Wayne State University (IACUC).

Targeted drug delivery: A1 reactive astrocytes were generated by culturing the purified astrocytes on PDMS coated tissue culture plates and then treating for 24 h with IL-1α (3 ng/ml, Sigma, 13901), TNF-α (30 ng/ml, Cell Signaling Technology, 8902SF), and C1q (400 ng/ml, MyBioSource, MBS143105). A2 reactive astrocytes were generated by culturing the purified astrocytes on PDMS coated tissue culture plates and then treating for 24 h with IL-1β (30 ng/ml, Cell Signaling Technology, 8900SF) and TNF-α (30 ng/ml, Cell Signaling Technology, 8902SF). A1 reactive astrocytes were targeted for 48 h using neutralizing antibodies to IL-1α (30 ng/ml, Abcam, ab9614), TNF-α (30 ng/ml, Cell Signaling Technology, 7321), and TGF-β (30 ng/ml, R&D Systems, 243-B3-002/CF). A2 reactive astrocytes were targeted for 48 h using neutralizing antibodies to IL-1P (30 ng/ml, Abcam, ab9722), TNF-α (30 ng/ml, Cell Signaling Technology, 7321), and IL-6 (30 ng/ml, Abcam, ab6672) (Liddelow et al., Nature. 541(7638):481-487, 2017; Guttenplan et al., Cell Rep. 31(12), 2020).

Polydimethylsiloxane (PDMS) coated tissue culture plates were prepared by mixing Sylgard-184 elastomer and curing agents at a ratio of 10:1 (w/v), then pouring into the plates and curing for 48 h.

Data presentation and statistical analysis: All data presented was performed using GraphPad Prism version 8. Two-tailed unpaired Student's t-test and two-way ANOVA test was performed. The mean with standard error mean (for the experiments done independently with technical replicates) and standard deviation was displayed. A predetermined significance level of P<0.05 was used in all statistical tests.

### Results

Understanding the inflammatory response following shunt implantation: Quantitative PCR (qPCR) was performed on collected tissue from failed shunts received from patients. Since the tissue obstructing shunts comprises of a more exaggerated astrocytic response, we investigated the up-regulation of the A1-specific reactive gene C3 and the A2-specific reactive gene EMP1. We observed that a heterogeneous up-regulation of both the A1- and the A2-specific reactive gene exist on both obstructed and non-obstructed shunts with no significant difference. In addition, we observed an increase in the EMP1 expression on non-obstructed shunts, while an increase in the C3 expression was noticed on obstructed shunts (FIGS. 2A-2B).

Indeed, A1 reactive astrocytes are a major source of the classical complement cascade component C3, however, other inflammatory cells in the tissue on obstructed shunts also induce the expression of C3. The increased expression of C3 on obstructed shunts is in accordance with other studies linking persistent neuroinflammation to neurodegeneration and adverse effects on the neural circuits and decrease excitatory neuronal function (Clark et al., Neurochem Res. 44(6):1410-1424, 2019). This is to recruit additional immunocytes to the site and exacerbate the secondary insult response.

Astrocytes are the dominant cell type bound directly to non-obstructed shunts and play a neuroprotective role, particularly in the acute phase of injury following an immediate disruption of the blood-brain barrier (BBB). Therefore, the increased expression of EMP1 on non-obstructed shunts is in accordance with other studies (Hanak et al., J Neurosurg Pediatr. 18(2):213-223, 2016).

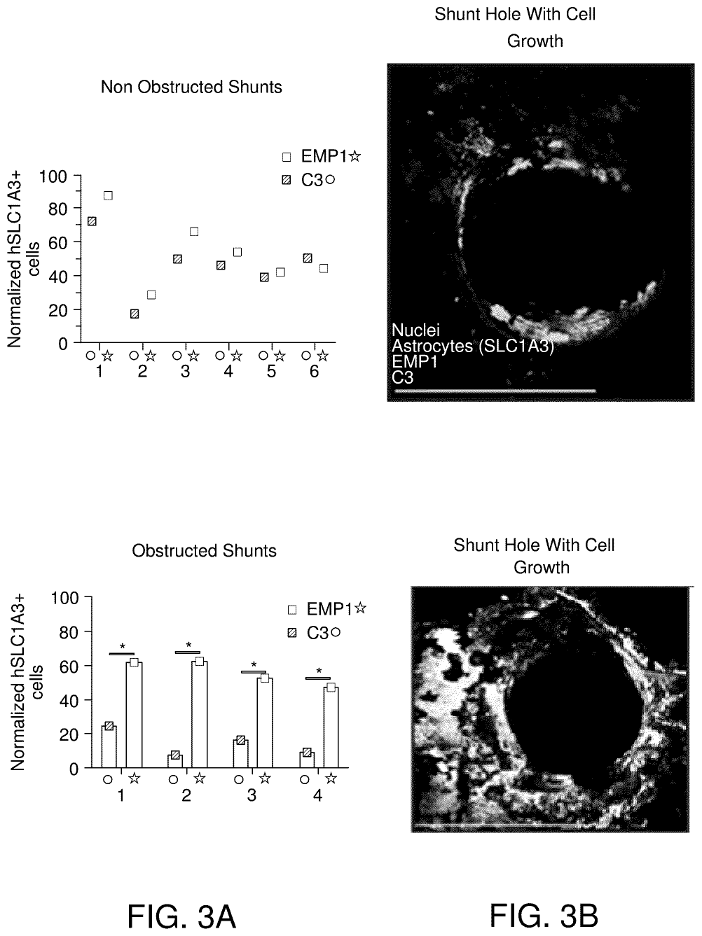

Understanding the astrocyte phenotype expression on implanted shunts: RNAscope fluorescent in situ hybridization was performed on collected tissue from failed shunts received from patients. In accordance with the presented qPCR data, it was found that the majority of SLC1A3+ astrocytes express A1 (C3) and/or A2 (Emp1) markers, suggesting that astrocytes can express a combination of A1 and A2 genes on shunt surfaces. In particular, it was observed that a greater number of SLC1A3+ astrocytes expressed the A2-specific gene Emp1 on both obstructed and non-obstructed shunts. Interestingly, the number of A2 reactive astrocytes are significantly larger on obstructed shunts compared to A1 reactive astrocytes (FIGS. 3A-3B). In recent work, astrocyte markers in obstructive masses were observed to be co-localized with proliferative markers, indicating that astrocytes are active on the shunt surface: they produce inflammatory cytokine IL-6 and proliferate. Since A2 reactive astrocytes are proliferative (Liddelow & Barres, Immunity. 46(6):957-967, 2017), they are responsible for the glial scar formation observed on obstructed shunts. This is in accordance with other studies, indicating that glial scar borders are formed by newly proliferated, elongated astrocytes that interact to corral inflammatory and fibrotic cells via STAT3-dependent mechanisms (Wanner et al., J Neurosci. 33(31):12870-12886, 2013), and that astrocytes in scar formation seem to be devoid of C3 upregulation (Duan et al., Proc Natl Acad Sci USA. 112(43), 2015).

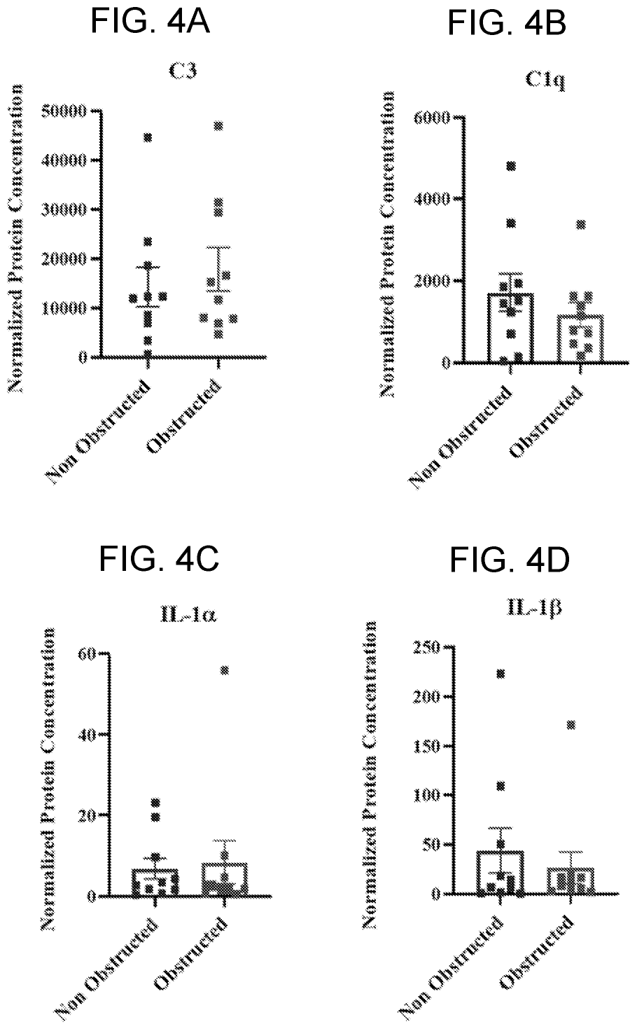

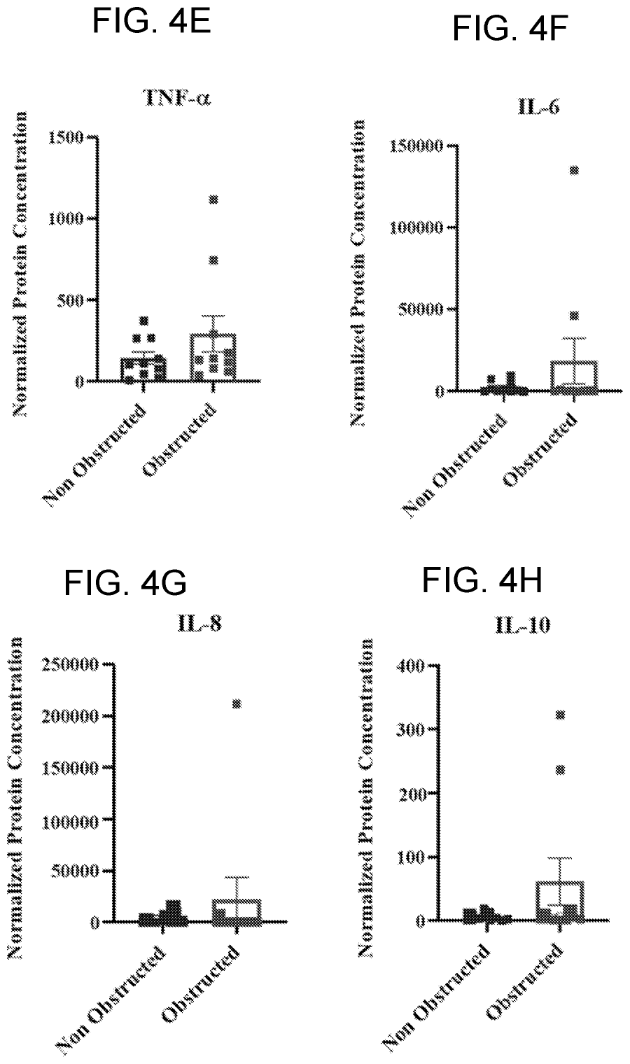

Cerebrospinal fluid biomarkers of neuroinflammation in obstructed and non-obstructed shunts: Using multiplex ELISA, this study investigated shunt failure through the CSF protein concentration profiles of select pro-inflammatory and anti-inflammatory cytokines for obstructed and non-obstructed shunts. C1q, IL-1α, and TNF-α induce A1 reactive astrocytes, IL-1β, TNF-α and IL-6 induce A2 reactive astrocytes. C3 is an A1 astrocyte marker. IL-8 and IL-10 inflammatory cytokines are of interest as they consistently stand out by being elevated in the CSF of hydrocephalus patients. Remarkably, in accordance with our qPCR data, we found higher neuroinflammation for obstructed shunts, however, with no significant difference compared to non-obstructed shunts confirming the heterogeneous mixed population of both the A1 and the A2 reactive astrocytes. Interestingly, higher levels of IL-6 are observed for obstructed shunts compared to non-obstructed shunts (FIGS. 4A-4B). As indicated in the RNAscope fluorescent in situ hybridization results, IL-6 primarily activates A2 reactive astrocyte proliferation through a positive feed-forward loop, activating local astrocytes to maintain the glial scar formation on the shunt surface.

In our recent paper, higher levels of IL-6 are observed for non-obstructed shunts compared to obstructed shunts (Harris et al., Fluids Barriers CNS. 18(1):1-14, 2021). However, obstructed and non-obstructed shunts were characterized based on the symptoms of obstruction defined by the patient's charts instead of the degree of actual tissue blockage on the shunt surface as described in this study.

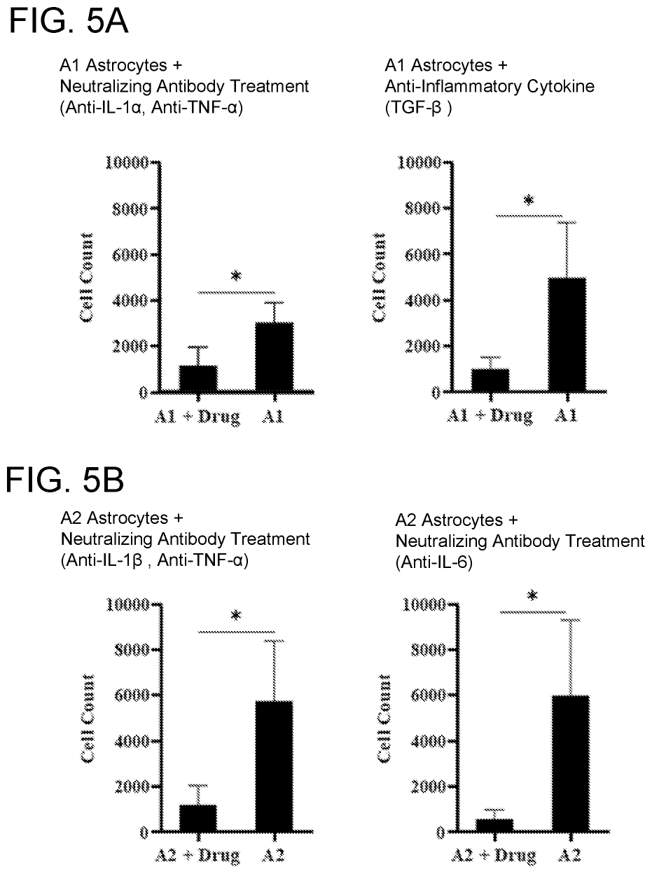

Inhibiting astrocyte cell activation and attachment on the shunt surface with neutralizing antibody treatment and anti-inflammatory cytokines: Now we understand that a heterogeneous mixed population of both the A1 and A2 reactive phenotype exist on the shunt surface. In addition, TNFα, IL-1α combined propel resting astrocytes into an A1 reactive phenotype (Liddelow et al., Nature. 541(7638):481-487, 2017) and co-stimulation with TNF-α and IL-1β induces an A2 reactive astrocyte phenotype (Hyvarinen et al., Sci Rep. 9(1):1-15), 2019. Also, IL-6 induces astrogliosis and astrocyte proliferation (Khodadadei et al., Commun Biol. 4(1):1-10, 2021). Therefore, we investigated whether the activity of astrocytes could be significantly reduced by simply employing already FDA-approved antibody therapies that inhibit human TNF-α, IL-1α, IL-1β, and IL-6. Hence, neutralizing antibodies to TNF-α, IL-1α, IL-1β and IL-6 were employed to decrease the activity of A1 and A2 astrocytes for a significant decrease in attachment on PDMS coated surfaces mimicking the shunt surface (FIGS. 5A-5B). These data are in accordance with other studies indicating that the knockout of reactive astrocyte activating factors slows disease progression (Guttenplan et al., Nat Commun. 11(1):1-9, 2020), dampening the formation of reactive astrocytes prevents neuronal death (Guttenplan et al., Cell Rep. 31(12), 2020), and astrogliosis inhibition attenuates hydrocephalus (Ding et al., Exp. Neurol. 320:113003, 2019).

The anti-inflammatory cytokine TGF-β was able to reset A1 astrocytes to a non-reactive state, significantly reducing cell attachment on the PDMS coated surface. This is in accordance with other studies indicating that TGF-β suppresses A1 astrocyte activation (Liddelow et al., Nature. 541(7638):481-487, 2017), reverses the formation of A1 astrocytes by fibroblast growth factor (FGF) signaling (Kang et al., Proc Natl Acad Sci USA. 111(29), 2014), and greatly reduces the expression of A1-specific markers (Gottipati et al., Acta Biomater. 117:273-282, 2020). Furthermore, TGF-β did not induce A2 reactive astrocyte attachment on the PDMS coated surface.

Taken together, these data suggest that drug therapies could be added to the shunt as device coating and released in vivo for enhanced next-generation medical devices.

### Discussion

This first-time study, which pulls strengths from the recent world-renowned Barres et al. study on astrocyte activation, represents a robust investigation of the changes in gene expression levels specific to astrocyte immune response following CSF shunt implantation. By shedding light on the mystery of astrocyte phenotype expression on shunt surfaces, root causes for shunt failure can be achieved to improve hydrocephalus treatment. FIG. 1 summarizes the microglia/macrophage and astrocyte reactions following neuroprosthetic device implantation.

Cell adhesion is not necessary to drive the neuroinflammatory response and biomaterials that go beyond reducing cell adhesion alone but also incorporate improved attenuation of inflammation in the tissue surrounding the implanted device may be beneficial (Jorfi et al., J Neural Eng. 12(1), 2015). In extensive studies, chronically implanted neural implants with coatings were compared to that of identical uncoated devices. In vitro, the coated implant significantly reduced astrocyte and microglial adhesion by ˜95% (Winslow et al., Biomaterials. 31(35):9163-9172, 2010). A similar reduction of cell adhesion was observed following device removal after two, four or 24 weeks of implantation in rat cortical tissue (Gutowski et al., J Biomed Mater Res A. 102(5):1486-1499, 2014). Interestingly, no significant difference was observed in the neuro-inflammatory response or the level of neuronal loss surrounding the coated implant compared to uncoated devices. A persistent inflammation was observed surrounding both uncoated and coated implants. Furthermore, neuronal density around the implanted devices was also lower for both implant groups compared to the uninjured controls. As no cells were found adhered to coated implants upon removal, both coatings were still functioning at the endpoints studied.

Our recent paper also indicates that under higher shear stress, despite less cell attachment to the surface, a significant increase in IL-6 secretion is detected (Khodadadei et al., Commun Biol. 4(1):1-10, 2021). Our data support others, which identify a necessity for control of the degree of inflammatory cell activation for considerable enhancement of device performance within the brain, in contrast to the common implant failure of reduced cell adhesion on the device surface in vivo (Tomaszewski, J Neural Eng. 12(1):11001, 2015; Winslow et al., Biomaterials. 31(35):9163-9172, 2010; Gutowski et al., J Biomed Mater Res A. 1486-1499, 2013; Sommakia et al., Front Neuroeng. 7:1-8, 2014). The results described in this Example, in combination with previous studies, present a proof of concept that to have significant impact, strategies should implement a focus on attenuating the initial inflammatory cell activation instead of only aiming at reducing cell adhesion on the device surface. Such strategies include decreasing shear activation as a primary cause of device failure (Hermann et al., Front. Bioeng. Biotechnol. 6:1-17, 2018; Tomaszewski, J Neural Eng. 12(1):11001, 2015; Moshayedi et al., Biomaterials. 35(13):3919-3925, 2014; He & Bellamkonda, PMID: 201204399. Boca Raton (FL): CRC Press/Taylor & Francis; 2008. Chapter 6; Spencer et al., Sci Rep. 1-16, 2017; Marimuthu & Kim, Anal Biochem. 437(2):161-163, 2013; Harris & McAllister, Childs Nerv Syst. 27(8):1221-1232, 2011; Harris et al., Exp Neurol. 222(2):204-210, 2010; Harris et al., J Biomed Mater Res A. 97(4):433-440, 2011), and directly antagonizing the accumulation of pro-inflammatory cytokines via targeted therapeutic for TNF-α, IL-1α, IL-1β, and IL-6.

The challenges for targeted drug delivery to A1 and A2 reactive astrocytes will be determining the parameters for delivery, specifically, the onset point, the dosage, the duration, and the delivery vehicle for anti-inflammatory device coating in vivo. Cytokine responses were strongly upregulated within a day post implantation indicating cytokine targeting strategies need to be present at the site of implantation immediately following implantation. Also, given the complexity of molecules involved in the inflammatory response, many other potential molecular targets could be identified for therapeutic purposes. However, when employing such strategies, as increasing evidence has shown that many inflammatory molecular mediators in the CNS act like double-edged swords. Their effects do not occur in an all or-none fashion; rather, they are concentration dependent. Therefore, it should be kept in mind that the goal is to contain any excessive activation rather than to remove all activity.

### Conclusion

First-time revealing of astrocyte phenotype expression leads to (1) a precise understanding of cellular response mechanism to device implantation and a precise interpretation of failure in chronically indwelling neuroprosthetics, (2) a therapeutic window for more targeted therapies to inhibit astrocyte activation and attachment on the implant. Therefore, for significant reduction in device failure, drug therapy is used for inhibition of cytokines and therefore inhibition of cell aggregation for achieving stable and long-term functional outcomes.

### Example 2: Treatment of Hydrocephalus by Decreasing

### Inflammatory Cytokine Response Using GIT 27

Surgical insertion of a ventricular shunt initiates a cytokine response shown to play a role in shunt failure caused by obstruction. These pro-inflammatory and anti-inflammatory cytokines cause astrocytes, amongst others, to enter an activated state which causes an increase in attachment. 4,5-Dihydro-3-phenyl-5-isoxazoleacetic acid (GIT 27) is a reagent with immunomodulatory properties that acts by blocking the main signaling protein on astrocytes and microglia called toll-like receptor 4 (TLR-4).

In this example, the effect of GIT 27 on astrocytes was tested when used as a pre-treatment, simultaneous treatment, and post-treatment relative to shunt insertion, represented by the introduction of IL-1β or IL-10. Control, DMSO vehicle control, and GIT 27 treated sample groups were assayed for cell counts and cytokine concentration data.

Exposure of astrocytes to GIT 27 in suspension caused a decrease in cell attachment (though this decrease could be influenced by the exposure to DMSO). On the other hand, GIT 27 was the cause for the significant decrease in the concentration of the majority of cytokines. Comparisons of the three treatment times showed that pre-treatment with GIT 27 is most effective at decreasing cellular attachment, but post-treatment for decreasing cytokine concentrations.

Imbalance of cerebrospinal fluid (CSF) production and absorption often necessitates insertion of a ventricular shunt in hydrocephalus patients (Pople, Neurol Pract. 73, 2002; Corns & Martin, Surgery. 30:142-148, 2012; Leinonen et al., Clin. Neurol. dx.doi.org/10.1016/B978-O-12-802395-2.00005-5, 2018; Harris et al., Fluids Barriers CNS. 12:1-15, 2015). Treatment using a shunt system to divert excess CSF has extremely high failure rates, with 30% failing within one year post surgery and 85% within 10 years (Riva-Cambrin et al., J Neurosurg Pediatr. 17:382-390, 2016; Shannon et al., Child's Nerv Syst. 28:2093-2099, 2012; Stone et al., J Neurosurg Pediatr. 11:15-19, 2013; Harris et al., J Biomed Mater Res—Part A. 97 A:433-440, 2011). Obstruction of the proximal catheter holes with microglia, macrophages, and astrocytes makes up 70% of these shunt failures (Harris et al., Fluids Barriers CNS. 12:1-15, 2015; Harris et al., J Biomed Mater Res—Part A. 97 A:433-40, 2011; Hanak et al., J Biomed Mater Res-Part B Appl Biomater. 106:1268-79, 2018; Hariharan et al., Fluids Barriers CNS. 18:1-12, 2021. doi.org/10.1186/s12987-021-00262-3; Chittiboina et al., J Neurosurg Pediatr. 11:37-42, 2013; Gluski et al., Fluids Barriers CNS. 17:1-10, 2020 doi.org/10.1186/s12987-020-00211-6). While newly emerging evidence suggests that some shunt obstruction is dependent on periventricular tissue contact, our biorepository of obstructed ventricular catheters show us gross variability across samples, luminal obstruction, clotting, and evidence of proliferation, suggesting that both tissue contact and growth are factors (Hariharan et al., Fluids Barriers CNS. 18:1-12, 2021. doi.org/10.1186/s12987-021-00262-3).

Inflammatory response following shunt insertion plays a major role in the failure rates, specifically obstruction due to increased cell attachment (Harris et al., Fluids Barriers CNS. 12:1-15, 2015). Pro-inflammatory and anti-inflammatory cytokines are the main signaling chemicals of the immunoinflammatory response within the body (Harris et al., Fluids Barriers CNS. 18:1-14, 2021. doi.org/10.1186/s12987-021-00237-4). Within the central nervous system, cytokines cause an increase in cell attachment due to the immunocompetent properties of astrocytes initiating astrogliosis (Hyvarinen et al., Sci Rep. 9:1-15, 2019). Central nervous system injury stemming from shunt insertion surgery transform astrocytes into its reactive phenotype (Deren et al., Exp Neurol. 226:110-119, 2010. doi.org/10.1016/j.expneurol.2010.08.010). Microglia and astrocytes contain a protein called toll-like receptor 4 (TLR-4), which aid in the regulation of the immune response by controlling signaling between cells (Zacharowski et al., Proc Natl Acad Sci USA. 103(16):6392-6397, 2006. doi.org/10.1073/pnas.0601527103; Pascual-Lucas et al., J Neurochem. 129:448-462, 2014).

Various methods have been developed to improve failure rates associated with shunt insertion by focusing on decreasing cell attachment and inflammation immediately surrounding the device responsible for the foreign body response. Utilizing coatings such as N-acetyl-L-cysteine, trypsin, and poly(2-hydroxyethyl methacrylate) have been shown to decrease cell adhesion (Hanak et al., J Biomed Mater Res-Part B Appl Biomater. 106:1268-1279, 2018; Al-Saloum et al., J Biomed Mater Res-Part B Appl Biomater. 2021; 109:1177-87; Achyuta et al., Langmuir. 26:4160-4167, 2010). These methods have shown the potential to decrease obstruction-dependent shunt failure. Other methods tested to regulate a more global inflammatory response utilize compounds such as polydopamine, hyaluronic acid, and decorin (Xue et al., ACS Appl Mater Interfaces. 9:33632-33644, 2017; Botfield et al., Brain. 136:2842-2858, 2013).

4,5-Dihydro-3-phenyl-5-isoxazoleacetic acid (GIT 27) is an immunomodulatory agent that works by reducing the production of pro-inflammatory cytokines (Stojanovic et al., Clin Immunol. 123:311-323, 2007; Laird et al., Glia. 62:26-38, 2014). GIT 27 has been shown to target TLR-4, and in doing so interferes with the secretion of tumor necrosis factor alpha (TNF-α) and consequently decreases concentrations of interleukin (IL)-1β, IL-10, and interferon gamma (IFN- [symbol] ) (Stojanovic et al., Clin Immunol. 123:311-323, 2007). Previously, it has been shown that 50 mg/kg of GIT 27, also known as VGX-1027, decrease the inflammatory response by inhibiting TLR-4 which in turn significantly reduced edema when administered as a four hours post-treatment (Laird et al., Glia. 62:26-38, 2014). Astrocytes, macrophages, microglia, and neurons are major sources of TLR-4 (Shi et al., Neuropharmacology. 145:259-267, 2019. doi.org/10.1016/j.neuropharm.2018.07.022).

In this Example, the response of astrocytes treated to GIT 27 was tested by analyzing total cell counts and cytokine concentration measurements using an enzyme-linked immunosorbent assay (ELISA). GIT 27 was administered as a pre-treatment, simultaneous treatment, or post-treatment, with respect to catheter insertion (that is, where catheter insertion is the “treatment”). This insertion was represented by exposing the samples to either IL-1β or IL-10 in the culture media, mimicking the immune response associated with catheter insertion. We hypothesized that GIT 27 treatment will cause a decrease in both cell count and cytokine concentrations to determine its utility as a surface or additive imbedded to the silicone shunt catheter or injection to reduce failure rate due to obstruction.

### Methods and Materials

PDMS Catheter Creation: Catheter samples were made by creating a polydimethylsiloxane (PDMS, silicone) solution, Sylgard 184 (Dow Corning), at a ratio of 10:1 elastomer to curing agent. This solution was then homogenized and placed into a degasser to remove bubbles. Once all the air bubbles were removed, 200 μL of the PDMS solution was pipetted into a 24 well plate to create flat disk samples for cell culturing. These samples were then degassed again to ensure were no bubbles in the disks and left for 48 hours to cure on a flat substrate.

Media Creation: A media stock solution was created by combining astrocyte media (ScienCell), supplement kit, 10 mL fetal bovine serum (FBS), 5 mL penicillin/streptomycin, and 5 mL astrocyte growth serum (ScienCell), and an additional 10 mL FBS (ScienCell). IL-1β and IL-10 were diluted to a concentration of 50,000 ng/mL and 100,000 ng/mL, respectively, in phosphate buffer solution (PBS, w/v). GIT 27 was dissolved in a dimethyl sulfoxide (DMSO) vehicle (Sigma-Aldrich) to a concentration of 20 mg/mL (w/v).

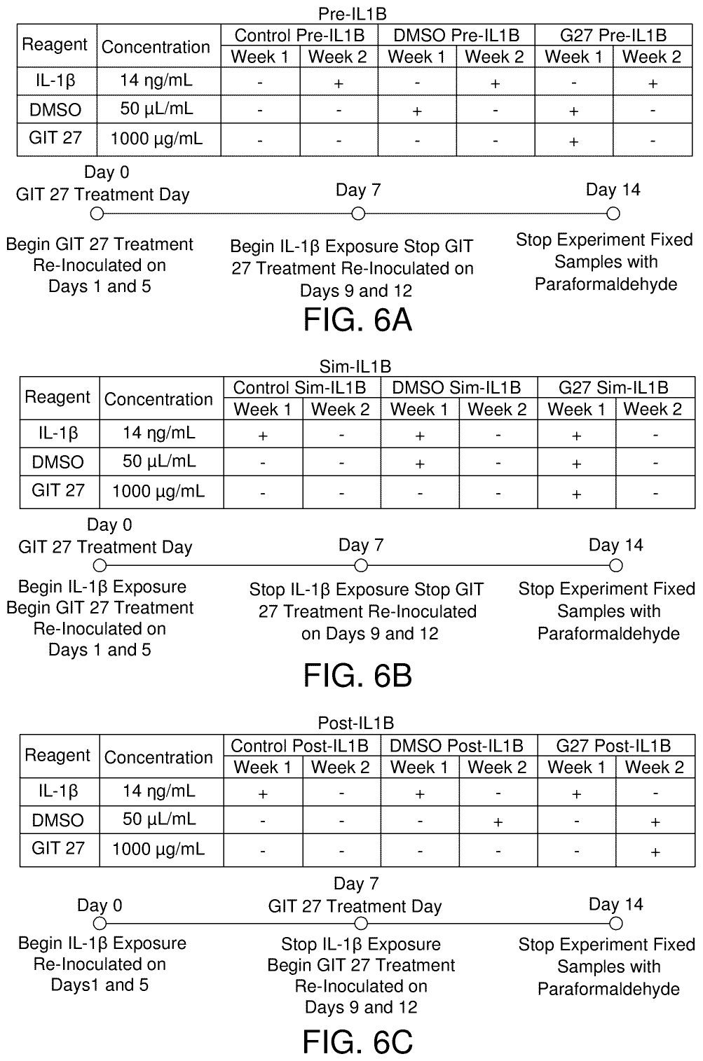

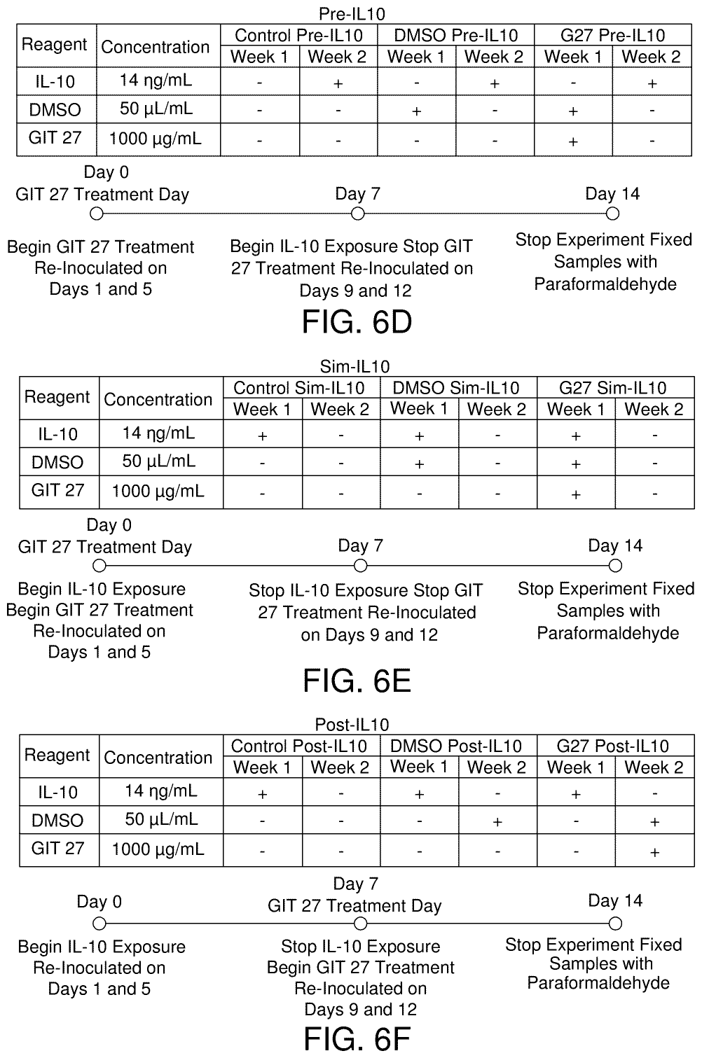

Media solutions for the different groups were made directly before each media change. Cytokines IL-1β or IL-10 were added to the media stock solution for a final concentration of 14.67 ng/mL. GIT 27 was added for a concentration of 1000 μg/mL in the media stock solution for our experimental group. Since GIT 27 is only soluble in DMSO, which has cytotoxic properties, a vehicle treatment group was performed to determine if it influenced cell counts or cytokine concentrations. Our vehicle group contained 50 μL/mL DMSO alone, which is proportional to the amount in the GIT 27 treated group. Concentrations were then made for evaluation of dose and time dependency (FIGS. 6A-6F).

Cell Culture: Human astrocyte cells were initially grown in T75 flasks with media changes every three days using basic astrocyte media and washed with Hanks Balanced Salt Solution without Calcium and Magnesium (Gibco). Once the flasks came to confluency, the cells were trypsinized using Trypsin-EDTA at 0.25% (Gibco) and seeded at a seeding density of 250,000 cells per well. These samples were broken up into three treatment groups: control, DMSO, and GIT 27 tested as a pre-treatment, simultaneous treatment, and post-treatment (FIGS. 6A-6F). Each group and treatment time were then either exposed to IL-1β or IL-10 separately at concentrations described for media creation.

GIT 27 Release Experiment: GIT 27 attachment to the surface of a shunt was completed using techniques similar to those described in previous work (Harris et al., J Biomed Mater Res-Part A. 98 A:425-33, 2011). Briefly, ventricular shunts (Medtronic 27600) were cut into 1.5 cm segments and placed in the plasma etcher. Immediately after finishing the etching cycle, the samples were submerged in a solution made up of 2.5 mg/mL N-hydroxysuccinimide (NHS), 2.5 mg/mL 1-Ethyl-3-(3-dimethylaminopropyl) carbodiimide (EDC), and GIT 27 at either a 5, 50, and 1000 μg/mL concentration. Samples were left to incubate for 24 hours and then washed with 0.9% (w/v) saline solution. Samples were then incubated in 0.9% saline solution at 37° C. to allow any GIT 27 to detach from the surface and erode into the saline solution.

At days 7, 14, 21, and 28, absorbance values were measured using the same methods as described in previous work (Al-Saloum et al., J Biomed Mater Res-Part B Appl Biomater. 109:1177-1187, 2021). Briefly, 2 μL of solution were placed on the nanodrop machine and measured using the a280 nanodrop setting on the Nano-Drop 2000 program. Absorbance values were recorded and compared to a standard curve made up of 7 concentrations evenly distributed between 0 and 1000 mg/mL of the GIT 27 in saline solution. The equation obtained from the standardized curve was y=18.7x+0.131, where y is the concentration of GIT 27 in the saline solution and x is the absorbance value.

Delivery Method: Following the nanodrop experiment, it was determined that the minimum concentration necessary to attach GIT 27 to the surface of the PDMS was 1000 μg/mL to display any attachment and release from the catheter. Analysis of the most effective delivery method was done by treating astrocytes with GIT 27 either attached to the surface PDMS or in suspension within the cell culture media. Using this concentration for sufficient surface coating, human astrocyte cells were cultured onto PDMS discs coated with GIT 27. Samples were also tested by adding GIT 27 at various concentrations in suspension within the cell culture media. Attached GIT 27 samples were conducted at concentrations 1000 and 3000 μg/mL, and for the in suspension concentrations 5, 50, and 1000 μg/mL in astrocyte media were used.

Immunofluorescence was performed using glial fibrillary acidic protein (GFAP) and 4′,6-diamidino-2-phenylindole (DAPI) solution (ThermoFisher). GFAP primary and secondaries were diluted to a ratio of 1:1000 and 5:1000, respectfully, in 0.4% Triton-X in PBS (w/v). Samples were incubated for 24 hours in each solution. After rinsing the samples with PBS, the samples were incubated in a 1:1000 DAPI solution in 0.4% Triton X solution for 30 minutes. Samples were then imaged submerged in PBS using a confocal microscope at 20× magnification and analyzed using Imaris software.

Cell Count Collection: Images were taking using a microscope (Fisher Scientific) and microscope attachable digital camera following each media change on days 1, 5, 7, 9, 12, and 14. These images were taken at approximately the center of each 24 well plate well with an area of 5.9788 mm². Counts were obtained using the FIJI software by converting the images to 32-bit. Contrast was enhanced to the value of 0.5 and background was subtracted to a value of 10. Finally, the threshold was set to 23-70 to obtain the cell count of each image.

ELISA: As previously published, the Bursky Center for Human Immunology & Immunotherapy Programs Immunomonitoring Laboratory at Washington University School of Medicine ran the multiplex assays according to the manufacturer's protocol (Khodadadei et al., bioRxiv 2021.11.04.467357, 2021. https://doi.org/10.1101/2021.11.04.467357). Samples of culture media analyzed were collected on days 1, 5, 7, 9, 12, and 14 of culture following seeding day. Each frozen supernatant media sample was rapidly thawed at 37° C. and centrifuged at 15,000 G for five minutes prior to incubating with the two-multiplex immunoassay for the following inflammatory cytokines: IL-1α, IL-1β, IL-6, TNF-α, IL-8, IL-10 (ThermoFisher Scientific), C3, and C1q (Millipore Sigma). Magnetic beads and assay buffer were added to all the wells of a 96 well-plate. Media samples and standards were then added in duplicate. The wells were thoroughly washed, and the detection antibody was then added followed by a streptavidin phycoerythrin incubation. Beads were resuspended with sheath fluid and 50 beads per region were acquired on a Luminex FLEXMAP3D system. The concentration of each analyte was then calculated by comparing the sample mean fluorescent intensity to the appropriate standard curve. Belysa v.1 software (Millipore Sigma) was used to generate a 5-parameter logistical curve fit algorithm. Protein concentration is reported as pg/mL for each analyte.

Statistical Analysis: Three conditions were tested in this experiment, treatment group, treatment time, and IL B or IL10 exposure, with a total of 16 different samples tested with an n=five of replicates. The three treatment groups tested were a control, vehicle control (DMSO), and GIT 27 (G27). Each treatment group was tested as a pre-treatment (denoted Pre-), dose simultaneous to treatment (denoted Sim-), and post-treatment (denoted Post-) as time points with respect to shunt insertion represented by exposure to either IL-1β (IL1B) or IL-10 (IL10). Utilizing the abbreviations described here we combined these to allow for an easier understand of the results presented. An example of this can be control Pre-IL1B, DMSO Pre-IL1B, and G27 Pre-IL1B which represents the three treatment groups when used as a pre-treatment to IL-1β exposure.

Cell counts were analyzed for every sample, total of 80 replicates, from the images taken following each media change (Table 1). Cytokine concentrations were obtained for a total of n=56 samples, Control 1 n=4, Control 2 n=4, DMSO n=24, GIT 27 n=24. Each sample was measured for IL-1β, IL-6, IL-8, IL-10, TNF-α, IL-1α, and IFN-γ concentrations. Concentrations for IFN-γ are not included since there was no detectable concentration in any of the treatment groups (Table 1).

TABLE 1
Percent change of average cell count and cytokine data for the
GIT 27 treated samples, compared to the DMSO treated vehicle

| | | Treatment | % Change Exposed to | |
|---|---|---|---|---|
| Data | | Time | IL-1β | IL-10 |
| | Cell | A | −15.09 | 22.97 |
| | Counts | B | 50.87 | −5.50 |
| | | C | 94.25 | −9.74 |
| Cytokine | IL-1β | A | −42.70 | 0.00 |
| Concentration | | B | −8.24 | −89.31 |
| | | C | −16.52 | 0.00 |
| | IL-6 | A | −76.60 | 21.96 |
| | | B | 31.97 | −28.89 |
| | | C | −28.57 | −76.82 |
| | IL-8 | A | −31.56 | −43.65 |
| | | B | −34.82 | −81.04 |
| | | C | −57.49 | −78.86 |
| | IL-10 | A | 26908.58 | −6.42 |
| | | B | −31.11 | −49.58 |
| | | C | −33.02 | −71.33 |
| | TNF-α | A | −12.41 | −13.92 |
| | | B | −8.65 | −53.95 |
| | | C | −33.45 | −80.45 |
| | IL-1α | A | −30.49 | 11.34 |
| | | B | −40.83 | −66.52 |
| | | C | −20.17 | −85.58 |

Key:
A = Pre-Treatment,
B = Simultaneous Treatment,
C = Post-Treatment

Normality was tested for both cell count and cytokine concentrations were done using the Anderson Darling equation for comparisons of treatment group and treatment time. Linear regression of the cell count data over time was used to determine time dependence.

Nonparametric analysis of the treatment group and treatment time of the cell counts and cytokine concentrations using the Kruskal Wallis Test with the Dunn's test with Bonferroni post-hoc test with α=0.05. One-way ANOVAs followed by a Tukey post-hoc test with α=0.05 was used for parametric results.

### Results

Time Dependence of Cell Counts: Analysis using a linear regression of average cell count over the 14-day time period revealed a significant change in slope compared to the x-axis, slope of zero, for G27 Post-IL1B (P=0.001) and DMSO Post-IL10 (P=0.001) samples. These two groups reach a maximum average cell count on day 5 and can be observed to be fully confluent in the images. Observations of the images taken on day 7 showed either a peeling or release of cells from the surface, and when compared to day 5 the average cell count is lower. The release or peeling of cells in these samples occurred between days 5 and 7 when they were yet to be treated with to any DMSO or GIT 27. All other samples expressed no significant change in slope.

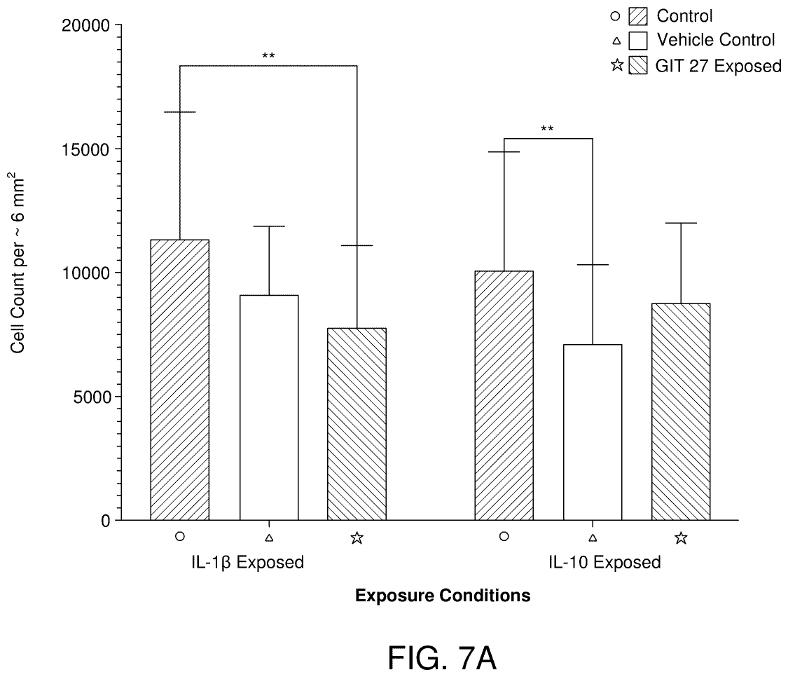

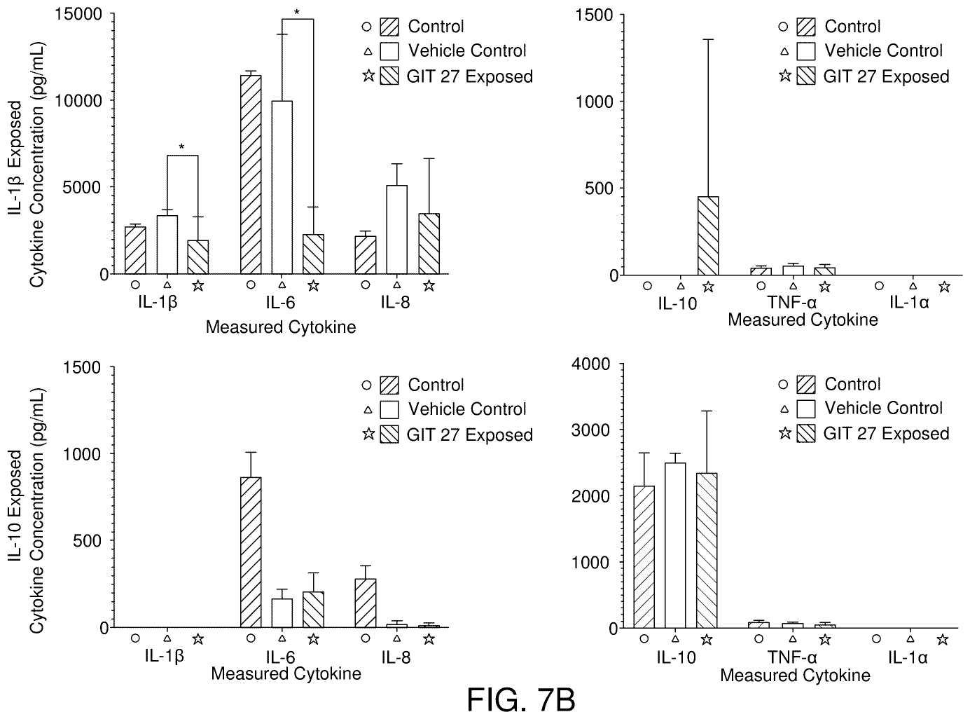

Comparison of Cell Counts by Treatment Group: The treatment groups exposed to IL-1p expressed a significant difference for all three treatment times Pre-IL1B (P=0.006), Sim-IL1B (P=0.001), and Post-IL1B (P=<0.0001) (FIGS. 7-9). G27 Pre-IL1B treatment group expressed a significantly lower cell count value when compared to control Pre-IL1B and insignificantly lower compared to DMSO Pre-IL1B groups. When comparing DMSO Sim-IL1B and DMSO Post-IL1B treatment groups there was a significantly lower number of cells compared to their associated control and G27 treated samples. Pre-IL10 samples showed a significant difference (P=0.003) between treatment groups (FIGS. 7-9). DMSO Pre-IL10 had a significantly lower cell count when compared to control Pre-IL10 and insignificantly lower than G27 Pre-IL10. There was no significant difference between samples under Sim-IL10 and Post-IL10 treatment conditions.

Comparison of Cytokine Concentrations by Treatment Group: Pre-IL1B treatment groups expressed a significant difference in IL-1P (P=0.032) and IL-6 (P=0.036) concentrations (FIGS. 7A-7B). The IL-1β and IL-6 concentration for G27 Pre-IL1B is significantly lower than DMSO Pre-IL1B, but not when compared to the control Pre-IL1B. Concentrations of IL-8, IL-10, TNF-α, and IL-1α expressed no significant difference for Pre-IL1B treatment groups. Samples treated under Pre-IL10 conditions expressed no significant difference for any of the cytokine concentrations (FIGS. 7A-7B).

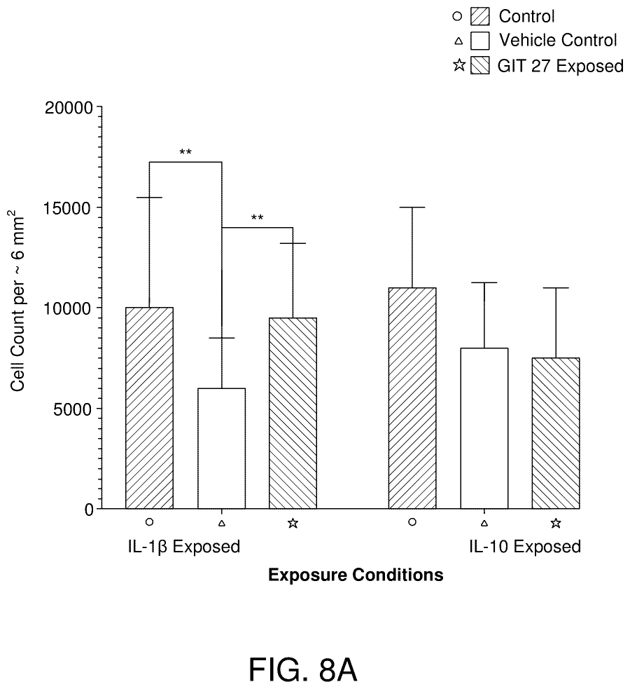

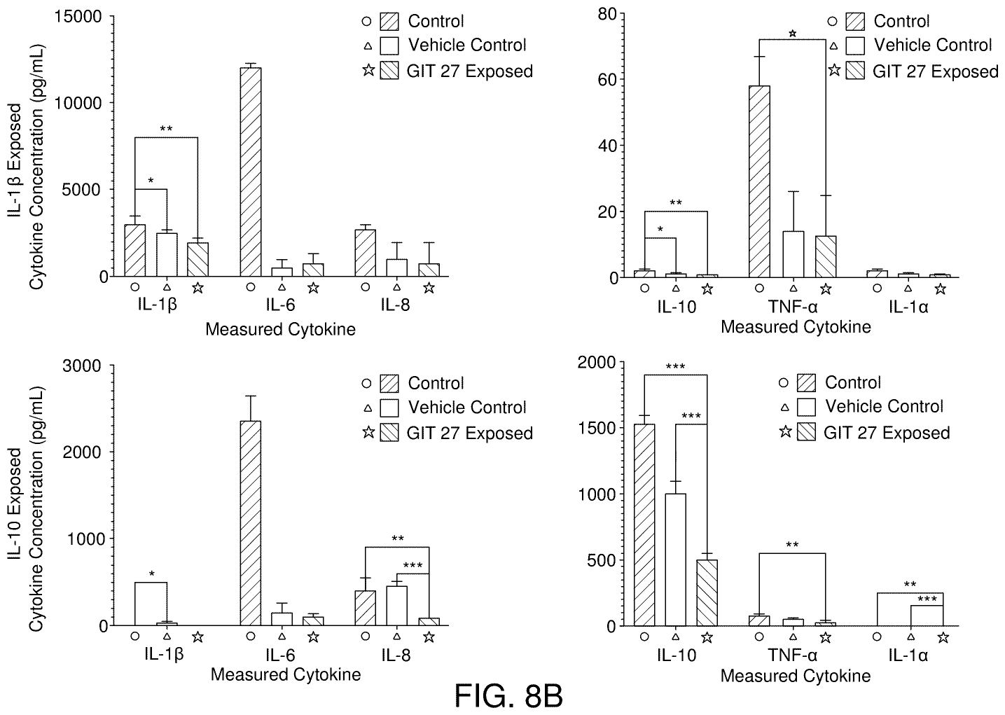

Cytokine concentrations of Sim-IL1B treatment groups expressed a significant difference in concentration of IL-1β (P=0.003), TNF-α (P=0.008), and IL-1α (P=0.026) (FIG. 8A-8B). The concentration of IL-1β and IL-10 is significantly lower for DMSO Sim-IL1B and G27 Sim-IL1B compared to control Sim-IL1B. IL-1α concentration are significantly lower when treated with G27 Sim-IL1B compared to the control Sim-IL1B. Sim-IL10 treatment groups reported a significant difference in IL-13 (P=0.019), IL-8 (p<0.001), IL-10 (p<0.001), TNF-α (P=0.020), and IL-1α (p<0.001) concentrations (FIG. 8A-8B). IL-1β concentrations is significantly lower for the control Sim-IL10 compared to DMSO Sim-IL10, and IL-1α concentration is higher compared to G27 Sim-IL10. The concentration of IL-8 and IL-1α are significantly higher for DMSO Sim-IL10 and control Sim-IL10 compared to G27 Sim-IL10 individually. G27 Sim-IL10 concentration of IL-10 were significantly lower compared to both DMSO Sim-IL10 and control Sim-IL10.

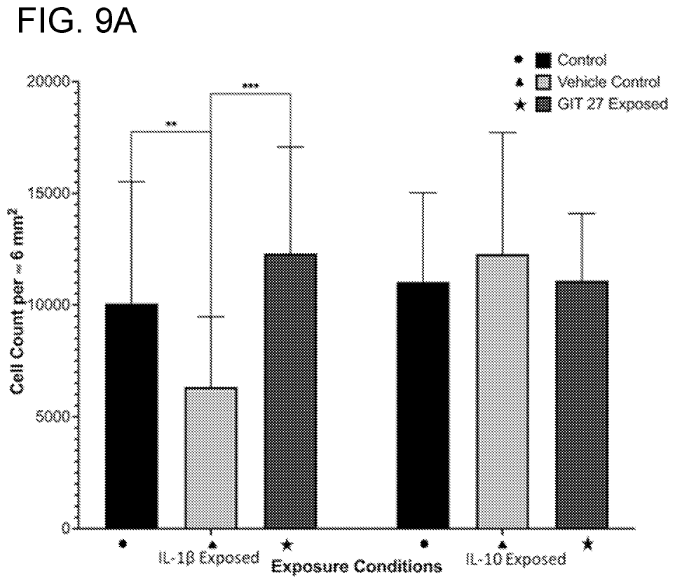

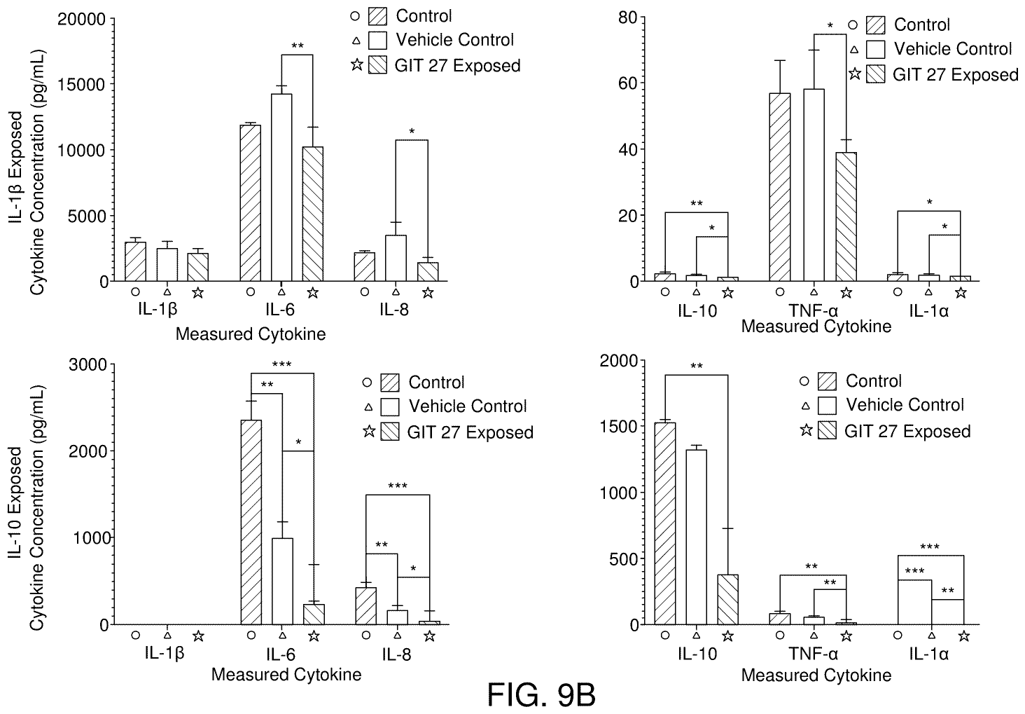

Under Post-IL1B conditions there was a significant difference in cytokine concentrations of IL-6 (P=0.003), IL-8 (P=0.011), IL-10 (P=0.004), TNF-α (P=0.034), and IL-1α (P=0.010) (FIGS. 9A-9B). There was a significantly lower concentration of IL-6, IL-8, and TNF-α for G27 Post-IL1B samples compared to DMSO Post-IL1B. Cytokine concentrations of IL-10 and IL-1α were significantly lower for G27 Post-IL1B compared to both control Post-IL1B and DMSO Post-IL B. The concentration of IL-6 (p<0.001), IL-8 (p<0.001), IL-10 (P=0.020), TNF-α (P=0.002), and IL-1α (p<0.001) were significantly different for Post-IL10 treatment groups (FIGS. 9A-9B). The concentration of IL-6, IL-8, and IL-1α for G27 Post-IL10 samples are significantly lower compared to the control Post-IL10 and DMSO Post-IL10. There is also a significantly lower concentration of these cytokines for DMSO Post-IL10 samples compared to the control Post-IL10. Concentration of IL-10 and TNF-α are significantly lower for G27 Post-IL10 compared to the control Post-IL10. In addition to this, there is also a significantly lower concentration of TNF-α of G27 Post-IL10 compared to DMSO Post-IL10.

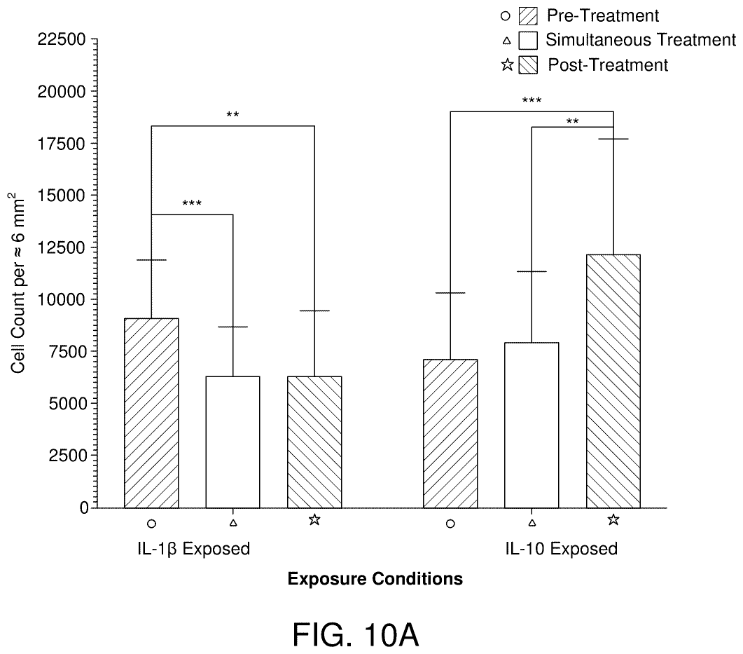

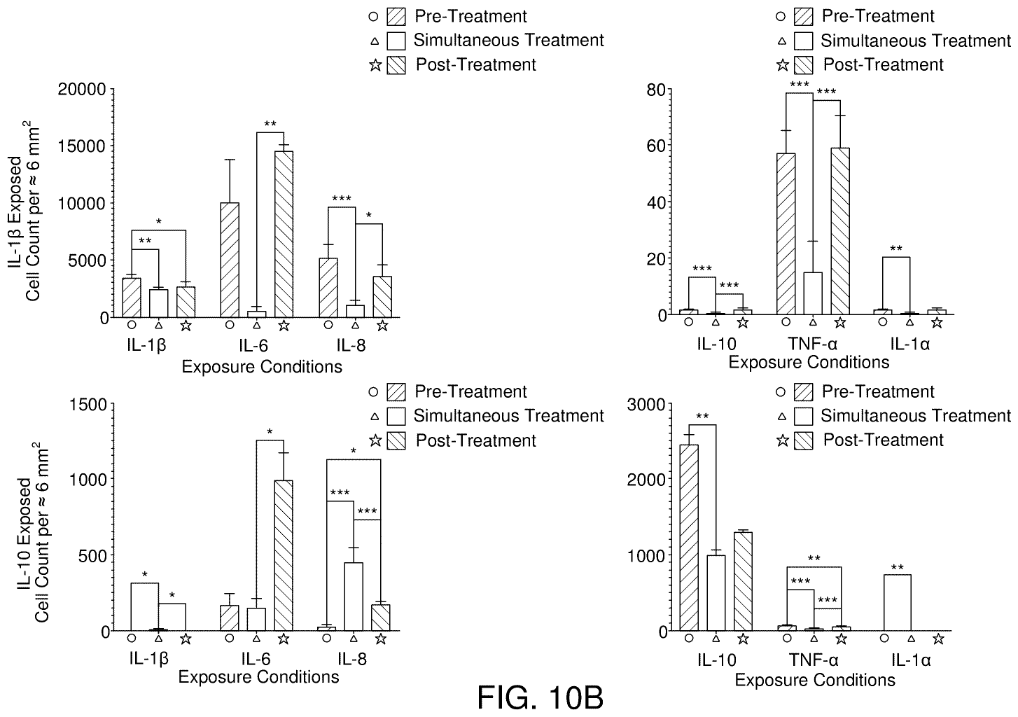

Comparison of Cell Counts by Treatment Time: Comparisons of treatment time for the control group exposed to either IL B or IL-10 expressed no significant difference and therefore is not included in this paper. The vehicle control treatment group showed a significant difference between treatment times when exposed to both IL B (p<0.001) and IL10 (p<0.001) (FIGS. 10A-10B). DMSO Sim-IL1B and DMSO Post-IL1B samples expressed a significantly fewer cells compared to DMSO Sim-IL1B, and DMSO Post-IL10 is significantly higher than DMSO Pre-IL10 and DMSO Sim-IL10. The GIT 27 treatment group expressed a significant difference in the cell count when exposed to both IL1B and IL10. G27 Pre-IL1B and G27 Pre-IL10 has significantly fewer cells compared to G27 Post-IL1B and G27 Post-IL10 respectively. There is also a significantly lower cell count for G27 Sim-IL10 samples compared to G27 Post-IL10.

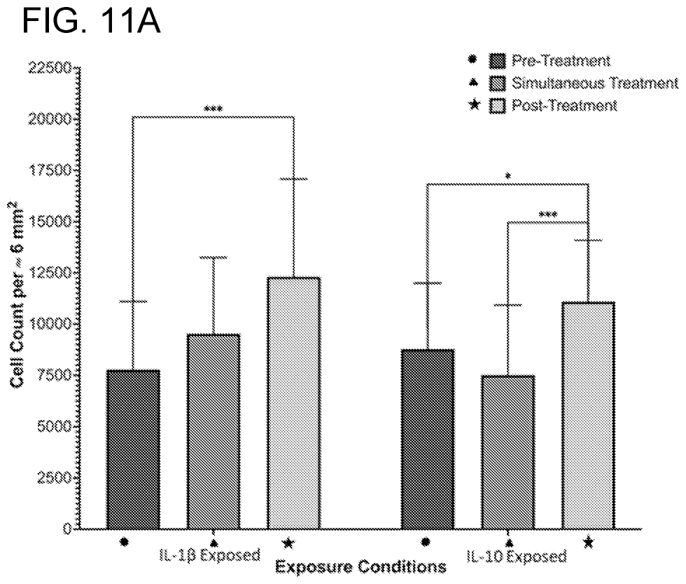

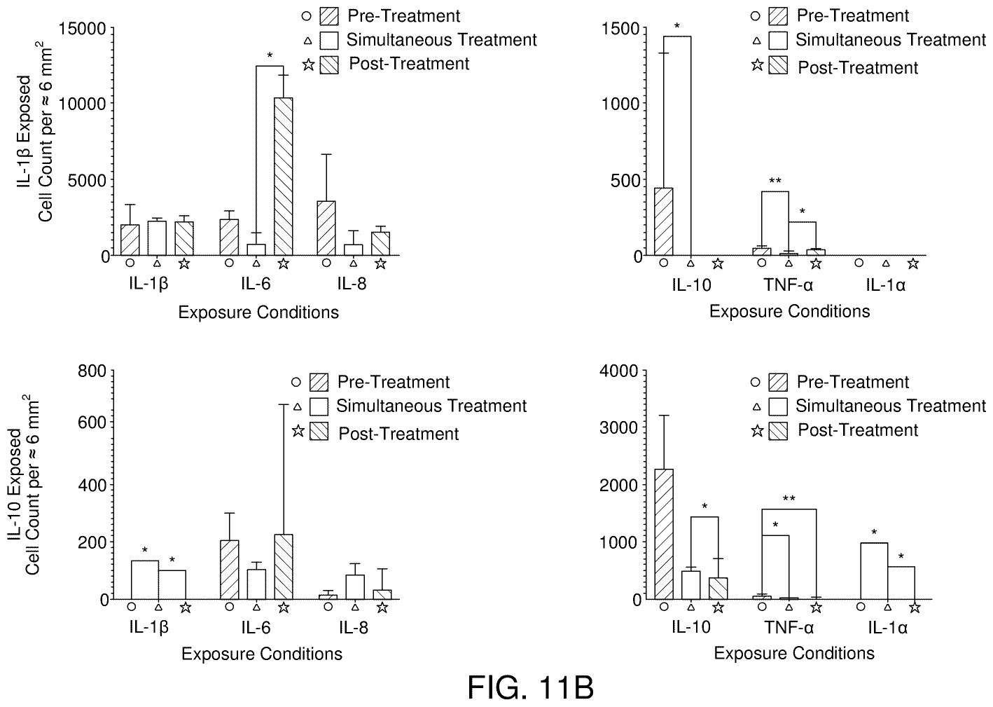

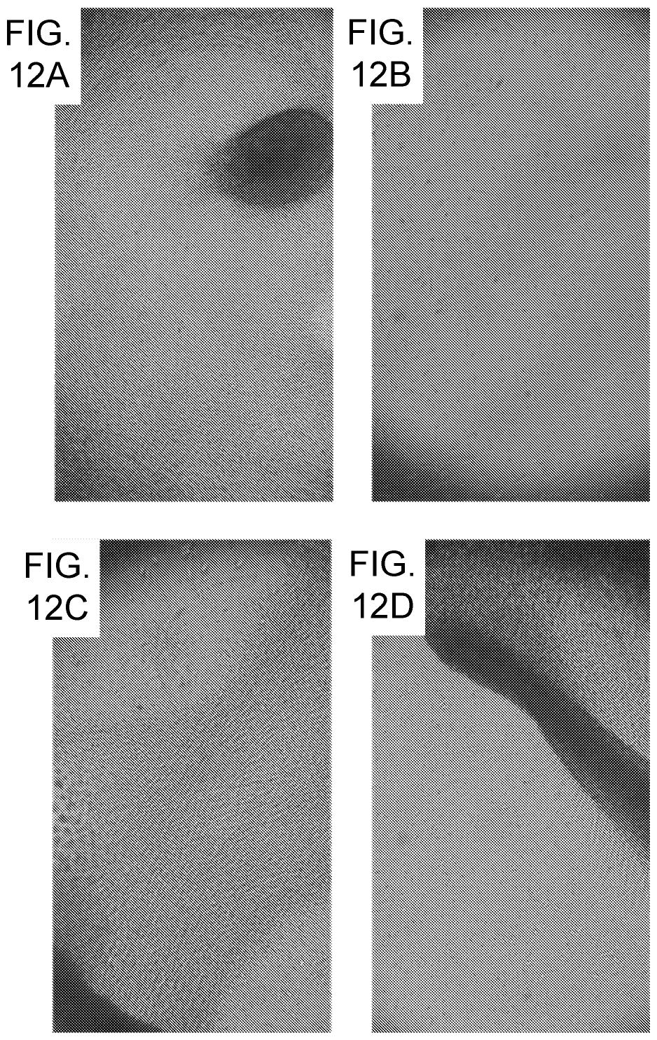

Comparison of Cytokines by Treatment Time: Treatment time comparisons of cytokine concentrations reported no significant difference for the control group exposed to IL1B or IL10 and is not included in this paper. Cytokine concentrations of the vehicle control group were significantly different for IL-1β (P=0.003), IL-6 (P=0.007), IL-8 (p<0.001), IL-10 (p<0.001), TNF-α (p<0.001), and IL-1α (p<0.001) when comparing the treatment times (FIGS. 11A-11B). DMSO Pre-IL1B has a significantly higher IL-1β concentration compared to both the DMSO Sim-IL1B and DMSO Post-IL1B. The concentration of IL-6 was significantly lower when under DMSO Sim-IL1B conditions compared to DMSO Post-IL1B, and DMSO Sim-IL1B is lower than the concentration of IL-1α of DMSO Pre-IL1B treatment times. DMSO Sim-IL1B has a significantly lower IL-8, IL10, and TNF-α concentration compared to both the DMSO Pre-IL1B and DMSO Post-IL1B treatment times.

GIT 27 treatment groups showed a significantly difference in treatment time for cytokine concentrations of IL-6 (P=0.015), IL-10 (P=0.023), and TNF-α (P=0.002) when exposed to IL1B, and when exposed to IL10 the concentrations IL-1β (P=0.005), IL-10 (P=0.018), TNF-α (P=0.004), and IL-1α (P=0.007) are significantly different (FIGS. 7A-7D). The concentration of IL-6 and TNF-α is significantly lower for G27 Sim-IL1B compared to G27 Post-IL1B, and lower for IL-10 and TNF-α compared to G27 Pre-IL1B. Concentrations of IL-1β and IL-1α are significantly higher for G27 Sim-IL10 compared to both G27 Pre-IL10 and G27 Post-IL10. G27 Post-IL10 concentration of IL-10 is significantly lower than the G27 Sim-IL10 group. TNF-α concentrations of G27 Pre-IL110 are significantly higher than the G27 Sim-IL110 and G27 Post-IL10 samples.

### Discussion

Pre-Experiments: Surface modification using GIT 27 had never been attempted before, so nanodrop analysis was conducted to determine a maximum concentration capable of coating a ventricular catheter and most effective delivery method. Determining this concentration, we ran an experiment to measure the absorbance values of the solution treated with 5 μg/mL, 50 μg/mL, and 1000 μg/mL of GIT 27. The lowest two concentrations, 5 μg/mL and 50 μg/mL, produced inconsistent values for both GIT 27 attached and released from the catheter. Data suggest that 1000 μg/mL GIT 27 following plasma etching physiosorbed the GIT 27 to the surface, of which was partially released over a four-week incubation period in saline.

Using this modification schema, we developed an in vitro model to test to determine the most effective GIT 27 delivery method, attached to PDMS surface or in suspension within the media, to decrease astrocyte attachment. Comparisons were made for the GIT 27 attached and in suspension samples to the unmodified control, plasma etched control, vehicle control, and in suspension samples exposed to plasma etching. Qualitatively, analysis of the immunofluorescent total cell count (DAPI) and astrocyte cell bodies (GFAP) treated with a 1000 μg/mL concentration of GIT 27 in suspension exhibited the lowest amount of DAPI and GFAP labeling compared to all other groups. Any sample exposed to plasma etching and treated with GIT 27 to attach the GIT 27 to the surface reported increased levels of GFAP compared to controls with no GIT 27 exposure. Since GFAP attaches to cells within their cytoplasm this increase may indicate an increase of cell debris attached to the PDMS surface.

Why Before, Same, After and Control, Vehicle, and GIT 27: These initial experiments indicated that the concentration 1000 μg/mL of GIT 27 in suspension within the cell culture media is the most effective treatment for decreasing astrocyte attachment. Utilizing this information, these conditions were used for the subsequent experiments. The response of astrocytes when treated with GIT 27 at three time points were examined by analyzing cell count and cytokine concentration of astrocytes cultures. All treatment groups were exposed to either IL-1B or IL-10 to represent shunt insertion. Control samples were not treated with GIT 27 and only underwent exposure to the shunt insertion cytokines. Treatment time was investigated to understand if pre-treatment, simultaneous treatment, or post-treatment of GIT 27 in suspension, where treatment represents shunt insertion (FIGS. 6A-6F).

Time Dependence and Cell Release: Results indicate that there is a significant time dependence for G27 Post-IL1B and DMSO Post-IL10, whereas all other groups have no time dependence. Qualitatively, we see these samples as overly confluent on day 5 then, on day 7, lacking cells after a large cell mass sloughed off the surface in these two groups. Since for the majority of the samples there is not time dependence, we were able to analyze the cell counts for the samples regardless of the time the counts were taken.

Treatment Group Comparisons: Cell count data for G27 Pre-IL1B reports a significantly lower number of cells compared to control Pre-IL1B. However, since the DMSO Pre-IL1B samples are insignificantly higher than G27 Pre-IL1B it cannot be determined that GIT 27 is the exclusive cause for the decrease (FIG. 7A). Concentration IL-1 and IL-6 were significantly lowered for G27 Pre-IL1B compared to the DMSO Pre-IL1B (FIG. 7B). Both of these cytokines are known to be associated with astrocyte activation, and these decreased levels could attribute to the insignificantly lower number of cells treated with to GIT 27 (Aschner, Toxicol Lett. 102-103:283-287, 1998). Even though insignificant, there was a trend showing increased IL-1β in DMSO Pre-IL1B compared to the control Pre-IL1B. This was potentially caused by the cytotoxic properties of DMSO and was somewhat negated by GIT 27 exposure. Cell counts of DMSO Pre-IL10 are significantly lower than control Pre-IL110, but not G27 Pre-IL10, which indicates that any decrease in cell attachment is probably caused by vehicle (FIG. 7A). Under these conditions there was no significant change in the concentration of the measured cytokines.

Analysis of Sim-IL1B and Post-IL1B revealed that DMSO is the reason for any cell loss that had taken place under these conditions (FIGS. 8A and 9A). Concentrations of IL-1 and IL-10 are also significantly lower for DMSO Sim-IL1B and G27 Sim-IL1B when compared to control Sim-IL1B. The lower number of cells when treated with DMSO, for both Sim-IL1B and Post-IL1B, compared to the controls and GIT 27 treated could attribute to the reduced level of these cytokines for these samples. Since the number of cells from G27 Sim-IL1B and G27 Post-IL1B is not significantly different to their associated controls, it seems as though GIT 27 may have blocked the cytotoxic effect. TNF-α concentrations are significantly lower for G27 Sim-IL1B samples in relation to the control Sim-IL1B. Since GIT 27 interference of TLR-4 signaling pathways, it will subsequently cause a decrease in the production of TNF-α (Stojanovic et al., Clin Immunol. 123:311-323, 2007). This decrease in TNF-α can also potentially contribute to the decrease in IL-1β concentrations for G27 Sim-IL1B as well.

DMSO Post-IL1B samples had an insignificant increase in cytokine concentration for IL-6, IL8, and TNF-α, but G27 Post-IL1B significantly lowered this value. Again, this may indicate that DMSO exposure can further exacerbate the cytokine response, although minor, but when paired with GIT 27 it neutralizes this negative effect. G27 Post-IL1B caused the concentration of IL-10 and IL-1α to be significantly lower compared to both control Post-IL1B and DMSO Post-IL1B. Decreased levels of TNF-α indicates that GIT 27 is effectively blocking TLR-4 which subsequently decreased the other cytokine levels.

Sim-IL10 and Post-IL10 samples expressed no significant difference between cells counts for the three treatment groups, indicating GIT 27 has no effect on the cell count (FIGS. 8A and 9A). GIT 27 caused the concentrations of IL-8, IL-10, and IL-1α to all significantly decrease for G27 Sim-IL10. These samples also significantly lowered the concentration of TNF-α compared to the control. Blocking of TLR-4 with GIT 27 will cause a decrease in TNF-α production, and a decrease in IL-8 and IL-10 will cause less signaling between cells causing a lower cytokine response. G27 Post-IL10 caused levels of IL-6, IL-8, IL10, TNF-α, and IL-1α to decrease. GIT 27 simultaneous treatment and post-treatment seems to have no effect on the cell count when exposed to IL10 but can decrease the cytokine response.

Treatment Time Comparison: Optimal treatment time of GIT 27 is an important aspect for the potential of using it to decrease the cytokine response. DMSO Pre-IL1B had the least amount of effect on cell count when compared to the other treatment times, indicating that the cytotoxic effect of DMSO is low when it is a part of a pre-treatment. Under G27 Pre-IL1B conditions, the cell count was significantly lower than Post-IL1B and insignificantly less than Sim-IL B. Therefore GIT 27 is most effective when Pre-IL1B, and in combination with DMSO's low effect as a pre-treatment it might be the best treatment time. DMSO Post-IL10 and G27 Post-IL10 caused the least amount of cell loss compared to the other treatment times. Since the other treatment time points had significantly lower cell counts for both DMSO and GIT 27 treated groups the current work does not identify the best treatment time when exposed to IL 0.

Greatest impact of cytokine concentration occurred when under DMSO Sim-IL1B conditions. Lower numbers of cells under these conditions could potentially have led to the lower concentrations of cytokines. Comparisons between GIT 27 treated samples indicate a generally lower concentration of cytokines when Sim-IL1B, excluding IL-1. DMSO Post-IL10 samples have increased levels of IL-6 and TNF-α which play a role in cell proliferation which probably can account for the significantly higher number of cells in Post-IL10. Further work will need to be done to determine the best treatment time for GIT 27 exposure.

### Conclusion

GIT 27 has immunomodulatory properties that interfere with the signaling process associated with TLR-4. In this study, it was determined that the most effective delivery method for treatment using GIT 27 is in suspension which had the least amount of cell attachment. Once the delivery method was determined we were able to develop an in vitro model to analyze the effect of GIT 27 treatment, at three time points, has on cell attachment and cytokine concentrations. Analysis of the treatment groups showed some decrease in cell counts, but we cannot completely determine that GIT 27 is the sole reason for any loss of cells. DMSO seems to have the potential to play a factor in the lower numbers, even if minor. On the other hand, we can determine that GIT 27 causes a majority of the decrease in the overall cytokine response. Even though we cannot fully determine that GIT 27 causes a decrease in cell attachment when comparing treatment times, we can determine that pre-treatment is the best time point because DMSO had the smallest effect and GIT 27 had the greatest effect on the count. GIT 27 post-treatment seems to have the largest effect while DMSO has the smallest effect on cytokine concentration. Future work should be done exposing both microglia and astrocytes to GIT 27 and the PDMS material to create a more physiological cytokine environment.

### Example 3: In Vivo Study of GIT 27 Activity

With the demonstration in Example 2 that GIT 27 is effective in an in vitro system to reduce cell attachment and pro-blockage cytokine production, it will be beneficial to characterize this activity in an in vivo model of implant biology, such as in in vivo model of hydrocephalus shunt treatment. There are a number of art-recognized in vivo models of ventricular shunts which can be used for this characterization. See, for instance, Iyer et al. (J Neurosur. 131(2):587-595, 2018; doi.org/10.3171/2018.1.jns172523); Di Curzio (Open J Mod Neurosur. 8(1):57-71, 2018; doi.org/10.4236/ojmn.2018.81004); Vandersteene et al. (Lab Animals. 52(5):504-515, 2018. doi.org/10.1177/0023677217753976), and so forth.

By way of example, the following experimental groups are tested, using an animal-based in vivo model for hydrocephalus and related shunt treatment:
sham control, no hydro
no pre-treatment+control catheter
GIT27 pre-treatment IP injection, no hydro
GIT27 pre-treatment IP injection+control catheter
GIT27 pre-treatment IP injection+GIT27 loaded/eluded catheter
no pre-treatment+GIT27 loaded/eluded catheter

Different amounts of GIT 27, and/or different times or manners of application, may be also be tested. It is expected that GIT 27 treatment will result in clinically positive results, including one or more of: reduced levels of pro-inflammatory cytokines (e.g., reduced levels of one or more of TNF-α, IL-1β, and/or IL-6), reduced cellular deposition (e.g., reduced deposition and clogging by astrocytes and/or glial cells), reduced glial scar formation, reduced blockage of catheters, maintained flow and drainage through the shunt system, and so forth.

This in vivo analysis can also be broadened to analyze other TLR-4 inhibitors, including those described herein.

### X. Closing Paragraphs

Specific descriptions provided herein and in the herewith filed documents are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; +19% of the stated value; ±18% of the stated value; +17% of the stated value; +16% of the stated value; ±15% of the stated value; +14% of the stated value; ±13% of the stated value; +12% of the stated value; +11% of the stated value; +10% of the stated value; ±9% of the stated value; ±8% of the stated value; +7% of the stated value; ±6% of the stated value; ±5% of the stated value; +4% of the stated value; ±3% of the stated value; +2% of the stated value; or +1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, references have been made to patents, printed publications, journal articles, published sequences and database entries, and other written text throughout this specification (referenced materials herein). Each of the referenced materials is individually incorporated herein by reference in their entirety for their referenced teaching.

It is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings and/or examples making apparent to those skilled in the art how the several forms of the disclosure may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. An implantable medical device comprising:
a coating comprising a biologically active agent over at least a portion of the surface of the device that is exposed to a biological fluid when the device is implanted, wherein the biologically active agent comprises:
a neutralizing antibody specific for TLR-4, IL-1β, IL-6, TNF-α, IL-1α, TLR2/6, IL-10, or IFN-γ;
an inhibitor of production or activity of TLR-4, TNF-α, IL-6, or IL-1β; or
an inhibitor of production or activity of TLR2/6, IL-10, IL-1α, or IFN-γ.

2. A medical implant that releases a biologically active agent in an amount effective to reduce or inhibit astrocyte and/or glia cell deposition associated with the medical implant, wherein the biologically active agent is selected from the group consisting of:
a neutralizing antibody specific for TLR-4, TNF-α, IL-1α, IL-1β, IL-6, TLR2/6, IL-10, or IFN-γ;
an inhibitor of production or activity of TLR-4, TNF-α, IL-6, or IL-1β; or
an inhibitor of production or activity of TLR2/6, IL-10, IL-1α, or IFN-γ, and wherein the medical implant comprises 0.1 μg to 1 mg of biologically active agent per mm² of surface area of the portion of the medical implant to which the biologically active agent is applied or incorporated.

3. The medical implant of claim 2, which comprises one or more of a tube, a chronic indwelling central nervous system (CNS) catheter, a neurological or neurosurgical device, a central nervous shunt, a pump, or a catheter.

4. A ventricular shunt coated with GIT 27 or another TLR-4 inhibitor.

5. An improved shunt catheter system, the improvement comprising including in the system at least one biologically active agent selected from the group consisting of:
a neutralizing antibody specific for TLR-4, TNF-α, IL-1α, IL-1β, IL-6, TLR2/6, IL-10, or IFN-γ;
an inhibitor of production or activity of TLR-4, TNF-α, IL-6, or IL-1β; or
an inhibitor of production or activity of TLR2/6, IL-10, IL-1α, or IFN-γ.

6. The improved shunt catheter system of claim 6, wherein the inhibitor of production or activity of TNF-α comprises one or more of: a neutralizing antibody specific for TNF-α, etanercept, infliximab, adalimumab, certolizumab pegol, or golimumab.

7. The improved shunt catheter system of claim 6, wherein the inhibitor of production or activity of IL-1α or IL-1β comprises one or more of: a neutralizing antibody for IL-1, a neutralizing antibody specific for IL-1α, a neutralizing antibody specific for IL-1β, anakinra, Rilonacept, or canakinumab.

8. The improved shunt catheter system of claim 6, wherein the inhibitor of production or activity of TLR-4 comprises one or more of: a neutralizing antibody of TLR-4, GIT 27, Eritoran, ibudilast, NI-0101, 1A6, or 15C1.

9. The improved shunt catheter system of claim 6, wherein the inhibitor of production of IL-6 comprises one or more of Tocilizumab, Sarilumab, Clazakizumab, Olokizumab, ALX-006, or 116-25.

10. A method of reducing cellular deposition and/or shunt device blockage in a fluid moving system, comprising introducing into the system:
a neutralizing antibody specific for TLR-4, TNF-α, IL-1α, IL-1β, IL-6, TLR2/6, IL-10, or IFN-γ;
an inhibitor of production or activity of TLR-4, TNF-α, IL-6, or IL-1β; or
an inhibitor of production or activity of TLR2/6, IL-10, IL-1α, or IFN-γ.

11. A method of reducing shunt device failure, comprising: inhibiting astrocyte and/or glia cell activation by contacting the shunt and/or a fluid passing over/through the shunt with:
a neutralizing antibody specific for TLR-4, TNF-α, IL-1α, IL-1β, IL-6, TLR2/6, IL-10, or IFN-γ;
an inhibitor of production or activity of TLR-4, TNF-α, IL-6, or IL-1β; or
an inhibitor of production or activity of TLR2/6, IL-10, IL-1α, or IFN-γ.

12. A method of mitigating astrocyte immune response to a chronically indwelling neuroprosthetic device, comprising:
inhibiting secretion and/or activity of at least one of TNF-α, IL-1β, or IL-6; or
inhibiting secretion and/or activity of TLR-4, TLR2/6, or IFN-γ.

13. The method of any one of claims 9-12, wherein the method inhibits formation or reverses formation of glial scar.

14. The method of any one of claims 9-12, wherein the method inhibits obstruction of one or more openings in the device.

15. A method of reducing astrocyte activation and attachment on a surface of a shunt, comprising contacting the shunt surface with a composition comprising:
a neutralizing antibody specific for TLR-4, TNF-α, IL-1α, IL-1β, IL-6, TLR2/6, IL-10, or IFN-γ;
an inhibitor of production or activity of TLR-4, TNF-α, IL-6, or IL-1β; or
an inhibitor of production or activity of TLR2/6, IL-10, IL-1α, or IFN-γ.

16. A method of reducing obstruction/blockage and failure of a ventricular shunt in a hydrocephalus patient, comprising contacting the shunt, before or after installation of the shunt in the patient, with a composition comprising:
a neutralizing antibody specific for TLR-4, TNF-α, IL-1α, IL-1β, IL-6, TLR2/6, IL-10, or IFN-γ;
an inhibitor of production or activity of TLR-4, TNF-α, IL-6, or IL-1β; or
an inhibitor of production or activity of TLR2/6, IL-10, IL-1α, or IFN-γ.

17. A method to decrease cellular attachment to a catheter surface, comprising contacting a surface of the catheter with a composition comprising:
a neutralizing antibody specific for TLR-4, TNF-α, IL-1α, IL-1β, IL-6, TLR2/6, IL-10, or IFN-γ;
an inhibitor of production or activity of TLR-4, TNF-α, IL-6, or IL-1β; or
an inhibitor of production or activity of TLR2/6, IL-10, IL-1α, or IFN-γ.

18. A method of decreasing astrocyte cell attachment on a medical implant device, comprising contacting the device with GIT 27 or another TLR-4 inhibitor before installation of the medical implant device.

19. A method of decreasing secretion of cytokines from astrocyte cells in a neurological implant system, comprising contact the astrocyte cells with GIT 27 or another TLR-4 inhibitor during or after installation of a neurological implant device.

20. A device, method, system, or composition for inhibiting or reducing cellular deposition on a medical implant, essentially as described herein.
